(19) United States
(12) Patent Application Publication
(10) Pub. No.: US 2013/0067616 A1
(43) Pub. Date: Mar. 14, 2013

(54) Plant Defensive Peptides

(71) Applicant: Svetlana Oard, Baton Rouge, LA (US)

(72) Inventor: Svetlana Oard, Baton Rouge, LA (US)

(21) Appl. No.: 13/583,834

(22) PCT Filed: Mar. 11, 2011
(86) PCT No.: PCT/US11/28026

§ 371 Date: Nov. 12, 2012

Related U.S. Application Data
(60) Provisional application No. 61/313,458, filed on Mar. 12, 2010.

(51) Int. Cl. A01H 5/00; A01H 1/02; C12N 5/10; A01H 5/10; C12N 15/29; C12N 15/82

(52) U.S. Cl. 800/265; 536/23.6; 435/320.1; 435/419; 800/279; 800/301

(57) ABSTRACT

A broad-range plant defense system employing thionins is disclosed. The invention protects various crop species and other plant species from attack by a wide range of phytopathogens, providing broad resistance to diverse plant diseases, using transgenes encoding a seed-derived thionin and a suitable signal peptide. Resistant crop varieties can not only prevent yield losses due to bacterial and fungal diseases, but can also expand the geographic boundaries of possible growing areas for economically important crops.

The benefit of the 12 Mar. 2010 filing date of U.S. provisional patent application Ser. No. 61/313,458 is claimed under 35 U.S.C. §119(e) in the United States, and is claimed under applicable treaties and conventions in all countries.

### TECHNICAL FIELD

This invention pertains to the use of defensive peptides in plants to protect against bacterial and fungal pathogens, including the expression of seed-derived thionins in leaves and other tissues to protect against fungal infection.

### BACKGROUND ART

Worldwide, crop losses totaling about 25-50% each year are attributed to diverse pests, including arthropods and microbial diseases. There is a continuing, unfilled need for improved methods for pest control and disease control in plants. Across various crop species, leaves are a primary target for many microbial pathogens and insects. Indeed, most of the major, yield-limiting plant diseases are foliar diseases. Well-known examples of serious fungal foliar diseases include powdery mildew, downy mildew, and rust in cereals, vegetables, and fruits. An effective, general means to reinforce the resistance of leaves to these diseases could have a dramatic effect on overall crop yields. Other important targets for disease in plants include roots, fruits, flowers, and other plant tissues.

Each crop species is typically susceptible to many different diseases and pests. In common practice, various synthetic crop protection compounds are applied to a crop at various times during the growing season to protect against different diseases and pests. These applications can exact high economic and environmental costs. For many diseases, there simply are no effective and economical chemical control measures. In such cases disease control, if available at all, depends primarily on disease-resistant plants. Disease-resistant crops can be especially valuable for developing countries, where the availability and affordability of crop protection compounds is limited. There is a continuing, unfilled need for new mechanisms for disease resistance in crops. A novel disease resistance mechanism that would provide protection against a broad range of diseases would be particularly valuable. A novel disease resistance mechanism that could be deployed in a broad range of different crop species would be particularly valuable.

Thionins are a class of highly basic, naturally occurring, antimicrobial peptides found in plants. Thionins exhibit broad and rapid activity against a variety of bacteria and fungi, with low minimal inhibitory concentrations. Thionins act directly on the cell membrane, a fact that slows the acquisition of resistance in pathogens. Examples of thionins include β-purothionin (βPTH) from wheat and α-hordothionin (αHTH) from barley, both of which are considered safe for human consumption. Both peptides contain nearly 20 cleavage sites that are recognized by trypsin or by pepsin, and the peptides are therefore quickly digested in the vertebrate gut. Furthermore, metal cation concentrations that are half of those typically seen in mammalian blood irreversibly inactivate thionins. Protection against bacterial pathogens has previously been reported in several plant species that have been engineered to express exogenous thionins. Despite the high antifungal activity of thionins in vitro, only partial protection against fungal pathogens has been reported to date. Despite extensive in vitro studies, the natural mechanisms by which plants mobilize thionins to inhibit bacterial and fungal pathogens are not well understood.

Thionins are excellent candidates for broad-range defense systems for crop protection. Antimicrobial peptides are important components of non-specific host defense systems and innate immunity in insects, amphibians, plants, and mammals, There are many antimicrobial peptides with antibacterial activity, but little or no antifungal activity. Thionins, on the other hand, have both broad spectrum antibacterial and broad spectrum antifungal activities. Because thionins act by permeabilizing microbial membranes, there is less likelihood that target microbes will develop resistance to these peptides,

Thionins appear to interact with phospholipids to cause membrane instability. Although the degree of inhibition of fungal or bacterial growth has been correlated with the strength of membrane permeabilizing activity, the detailed mechanism by which thionins act is not fully understood.

F. Terras et al., “Synergistic Enhancement of the Antifungal Activity of Wheat and Barley Thionins by Radish and Oilseed Rape 2s Albumins and by Barley Trypsin Inhibitors,” Plant Physiol., vol. 103, pp. 1311-1319 (1993) reported that thionins, which had primarily been considered to be storage proteins, also exhibited activity in inhibiting the growth of pathogenic fungi. Adding di- and monovalent metal ions at 1 and 50 mM concentrations, respectively, inhibited the lytic activity of the thionins.

D. Florack et al., “Expression of biologically active hordothionins in tobacco,” Plant Mol. Biol., vol. 24, pp. 83-96 (1994) discloses studies on the effects of the pre-sequences and pro-sequences on hordothionin expression, processing, sorting and biological activity and hence the feasibility of engineering bacterial disease resistance into crops.

P. Epple et al., “Overexpression of an Endogenous Thionin Enhances Resistance of Arabidopsis against Fusarium oxysponim,” The Plant Cell, vol. 9, pp. 509-520 (1997) reported that overexpression of Arabidopsis thionin under a constitutive promoter enhanced the resistance of Arabidopsis to the fungal pathogen Fusarium mysporum.

K. Thevissen et of., “Permeabilization of Fungal Membranes by Plant Defensins Inhibits Fungal Growth,” Applied and Environmental Microbiology, vol. 65, pp. 5451-5458 (1999) demonstrated a correlation between α-purothionin inhibition of fungal growth and membrane permeabilization in the fungi Neurospora crassa and Saccharomyces cerevisiae. A concentration as low as 0.5 82 M inhibited fungal growth by up to 50%. Membrane permeabilization significantly increased within 10 minutes after the addition of α-purothionin Adding 5 mM Ca⁺² considerably limited both growth inhibition and membrane permeabilization by α-purothionin.

P. Hughes et of., “The Cytotoxic Plant Protein, β-Purothionin, Forms Ion Channels in Lipid Membranes,” Journal of Biological Chemistry, vol. 275, pp. 823-827 (2000) reported data supporting the hypothesis that thionin's primary mode of action is to produce ion channels in cell membranes, which destroys essential ion concentration gradients. The formation of ion channels was observed both in artificial lipid bilayer membranes, and in the plasmalemma of rat hippocampal neurons. 10 mM Ca⁺² completely blocked thionin channel activity.

T. Iwai et al., “Enhanced Resistance to Seed-Transmitted Bacterial Diseases in Transgenic Rice Plants Overproducing an Oat Cell-Wall-Bound Thionin,” Molecular Plant-Microbe Interactions, vol. 15, pp. 515-521 (2002) reported transgenic rice in which a leaf-specific thionin gene from oat. Asthil, was overexpressed under a strong constitutive promoter. The thionin precursor contained a 28-residue signal peptide. high levels of oat thionin accumulated in the cell walls of 10-day-old coleoptiles. The transgenic rice was observed to be resistant to the bacterial pathogen Burkholderia plantarii.

Y. Chan et al., “Transgenic tomato plants expressing an Arahidopsis thionin (Thi2.1) driven by fruit-inactive promoter battle against phytopathogenic attack,” Planta, vol. 221, pp. 386-393 (2005) described a transgenic tomato plant in which Arabidopsis thionin Thi2.1 was expressed under the control of a promoter that was active in roots, and also incidentally active in leaves, but inactive in fruits. Significant levels of resistance were reported both against the bacterial pathogen Ralstonia solanacearurn and against the fungal pathogen Fusarium aysportun.

S. Oard et al., “Characterization of antimicrobial peptides against a U.S. strin of the rice pathogen Rhizoctonia solani,” J. Appl, Micro., vol. 97, pp. 169-180 (2004) reported tests with twelve natural and synthetic antimicrobial peptides in vitro. The wheat seed peptide purothionin showed strongly inhibitory activity against the fungal. phytopathogen R. solani, similar to that of the antifungal antibiotics nystatin and nikkomycin Z. Cecropin B, a natural peptide from the Cecropia moth, and the synthetic peptide D4E1 also had high inhibitory activity against R. solani. Membrane permeabilization levels strongly correlated with antifungal activity of the peptides.

S. Oard et al., “Expression of the antimicrobial peptides in plants to control phytopathogenic bacteria and fungi,” Plant Cell Reports, vol. 25, pp. 561-572 (2005) described experiments in which three antimicrobial peptides previously known to have in vitro antifungal activity were expressed in Arabidopsis to compare their activities in planta. β-Purothionin, cecropin and phor21 were expressed under an endogenous promoter with a moderate level of expression, and were excreted extracellutarly, The β-purothionin produced the greatest antibacterial and antifungal resistance. Cecropin B enhanced only antibacterial activity, while phor21 did not improve antimicrobial resistance. However, fusion of EGFP to the C-terminus of the thionin precursor rendered the mature thionin inactive.

A. Carlson et al., Barley hordothionin accumulates in transgenic oat seeds and purified protein retains anti-limgal properties in vitro. In Vitro Cell. Dev. Biol,—Plant 42: 318-323 (2006) discloses the genetic transformation of oat with barley hordothionin. The transgene was expressed in both leaf and seed tissue, but transgenic protein accumulated only in the seed. The authors speculated that “[i]f the protein could be retained in the leaf it may also serve as a transgenic form of resistance to leaf-based pathogens,” However, there was no suggestion for how to cause the exogenous thionin peptide to be retained in the oat leaf. By contrast, the authors reported a prior study by others reporting that mature HTH peptide had accumulated in tobacco, speculating that this might reflect differences between monocotyledonous and dicotyledonous protein targeting systems.

Little is presently known about the mechanisms by which plants mobilize thionins to inhibit bacterial and fungal pathogens. The plant plasmalemma is permeable by thionins. However, barley leaf-specific thionins accumulate in the cell wall, which requires passage through the plasmalemma without harming plant cells. See Bohlmann, H., Clausen, S., Behnke, S., Giese, H., Hiller, C., Reimann-Philipp, U., Schrader, G., Barkholt, V. and Apel, K. 1988. Leaf-specific thionins of barley—a novel class of cell wall proteins toxic to plant-pathogenic fungi and possibly involved in the defense mechanism of plants. EMBO J 7:1559-1565.

Fusion of green fluorescent protein (GFP) to the C-terminus of the acidic protein affects thionin antimicrobial activity in leaf tissues. See Oard, S., and Enright, F. 2006. Expression of the antimicrobial peptides in plants to control phytopathogenic bacteria. and fungi. Plant Cell Rep. 25: 561-572. While the C-terminus of the mature thionin participates in forming the global fold, the N-terminus is involved in the phospholipid-binding site of the mature thionin. Rao, U., Stec, B., and Teeter, M. 1995. Refinement of purothionins reveals solute particles important for lattice formation and toxicity. 1. alpurothionin revisited. Acta Crystallogn Sect. D D51: 904-913; Stec, B., Rao, U., and Teeter, M. M. 1995. Refinement of purothionins reveals solute particles important for lattice formation and toxicity. Part 2: Structure of beta-purothionin at 1.7 angstroms resolution. Acta Crystallogr, Sect, D 51: 914-924.

Cell-wall-bound thionins have been observed to accumulate in high concentrations at the penetration sites of a resistant barley cultivar following infection with the fungal pathogen that causes powdery mildew, but not in a susceptible barley cultivar. See Ebrahim-Nesbat, F., S. Behnke, A. Kleinhofs, and K. Apel, 1989. Cultivar-related differences in the distribution of cell-wall-bound thionins in compatible and incompatible interactions between barley and powdery mildew Planta 179:203-210. Overexpression of an endogenous thionin (encoded by the Thi2.1 gene in Arabidopsis) enhanced plant resistance to Fusarium oxysporum, See Epple, P., K. Apel, and H. Bohlmann. 1997, Overexpression of an endogenous thionin enhances resistance of Arabidopsis against Fusarium oxysporurn. Plant Cell 4:509-520. Expression of Thi2.1 in tomato enhanced resistance both to bacterial wilt and to Fusarium wilt. See Chan, Y, V. Prasad, K. Chen, P.Liu, M. Chan, and C. Cheng. 2005. Transgenic tomato plants expressing an Arabidopsis thionin (Thi2.1) driven by fruit-inactive promoter battle against phytopathogenic attack. Planta 221:386-393. Overproduction of an oat cell-wail-bound thionin in rice enhanced resistance to seed-transmitted bacterial diseases. See Iwai, T., H. Kaku, R. Honkura, S. Nakamura, H. Ochiai, T. Sasaki, and Y. Ohashi, 2002. Enhanced resistance to seed-transmitted bacterial diseases in transgenic rice plants overproducing an oat cell-wall-bound thionin. Mol Plant Microbe Interact 15:515-521. Silencing of a thionin gene (PR13/Thiortin) reduced antimicrobial resistance to Pseudomonas syringae pv. tomato DC3000 in a naturally resistant Nicotiana attenuata. See Rayapuram, C., J. Wu, C. Haas, and 1. Baldwin. 2008. PR-13/Thionin but not PR-1 mediates bacterial resistance in Nicotiana attenuata in nature, and neither influences herbivore resistance. Molecular Plant-Microbe Interactions 21:988-1000.

Following are presentations that are related to the invention described here, and that have given by the present inventor and colleagues: S. Oard et al., “Tuning exogenous expression of a wheat antimicrobial peptide purothionin,” abstract (published) and poster (unpublished), Keystone Symposium on Molecular and Cellular Biology, Plant Innate Immunity (Keystone, Colo., February 2008); S. Oard et al., “Plant antimicrobial peptides: Thionins as Nature's invention for weapons of mass protection,” presentation and abstract, 240th American Chemical Society National Meeting & Exposition (Boston, Mass., Aug. 26, 2010); and S. Oard et al., “Effects of signal peptide on transgenic expression of antimicrobial peptide hordothionin,” presentation and abstract, Receptors and Signaling in Plant Development and Biotic Interactions, (Tahoe City, Calif., Mar. 14-19, 2010). An audio recording of the Aug. 26, 2010 presentation is available: http://www.softconference.com/acschem/sessionDetail.asp?SID=226590. The complete disclosures of each of these presentations are incorporated by reference.

### DISCLOSURE OF THE INVENTION

I have discovered an improved, broad-range plant defense system employing seed-derived thionins in leaf tissues, root tissues, fruit tissues, flower tissues, and other plant tissues besides seeds. The invention provides various crops and other plant species with broad resistance to diverse plant diseases. This broad disease resistance will save substantial time and resources as compared to developing resistance for multiple individual pathogens one-by-one. Thionins have not previously proven practical for broad disease resistance in crops and other plants against diverse bacterial and fungal pathogens. Thionin-based disease resistance may be used as a reliable solution to increase food security. Resistant crop varieties can not only prevent yield losses due to bacterial and fungal diseases, but can also expand the geographic boundaries of possible growing areas for economically important crops, especially in cases where expansion has previously been limited by disease problems, Crops with improved disease resistance significantly reduce the costs for chemicals, and will also help the environment, Through the use of the invention growers can reduce or even eliminate dependence on pesticides.

Many, probably most seed-specific thionins are safe for consumption by humans, other mammals, and other vertebrates. A preferred thionin, α-hordothionin, is expressed natively in barley seeds, and is widely consumed from that source without toxic effects, Other safe and active thionins are known in the art, and include for example β-hordothionin, α1-purothionin, β-purothionin, other hordothionins, other purothionins, and avenothionins. Many other thionins are known in the art, and many more can readily be identified through standard genomic techniques. Commonly, there are many homologous thionins present even in the genome of a single plant species, presumably each adapted to combat different pathogens as part of the plant's evolving innate immune system. Any of these various thionins or their corresponding coding sequences can be identified, isolated, and sequenced using standard techniques, and used in the present invention.

An exogenous gene is introduced into a plant's genome to cause the expression of a seed-derived thionin in the leaf tissue. The thionin is excreted and cleaved to associate with the cell wall, so that the thionin does causes no significant damage to the host cell, Incorporation of a suitable signal peptide is important for expression in the leaves or other target tissue, and for direction to the proper cellular location, without damage to the host cells. The seed-derived thionin may be native to the same species as the transformed plant, or to another species.

Signal peptides (SPs) play an important role in regulating the activity of thionins in plant tissues. Without wishing to be bound by this hypothesis, I propose that the C-terminal motif of the signal peptide is especially important in regulating the activity of exogenous thionins in leaf cells during processing and transport. The central motif is hydrophobic, and is essential for excreting thionin outside the plasmalemma. The N-terminal motif is plant tissue-specific (but not necessarily species-specific), and causes the accumulation of biologically active thionin at levels sufficient to inhibit fungal growth. I have discovered a novel, preferred signal peptide that is particularly well suited for this function. The preferred signal peptide contains 27-28 amino acids, as compared to the 18-21 amino acids that are more typical for native seed-specific thionin signal peptides. The novel, preferred signal peptide is derived from the native thionin signal, fused at the amino terminus to a 7-10 amino acid sequence based in part on a segment of the signal sequence from oat thionin, and in part on a consensus sequence from thionins of several species. In an alternative embodiment, the signal sequence from a leaf thionin is fused with the active peptide portion of a seed thionin. More generally, each of the three motifs forming the signal peptide may natively all come from the same species or from different species, and may individually be from the same species as the transformed plant or from different species. Or a consensus sequence or modified consensus sequence may be used. The consensus sequence preferably includes a 4-10 amino acid residue N-terminus containing basic residue(s); a 10-14 residue hydrophobic central region; and a 2-7 residue C-terminus containing acidic and polar residues. Examples are shown as SEQ ID NOS. 5 through 9.

The coding sequence is operatively linked to an appropriate promoter. Examples of suitable promoters are known in the art and include constitutive promoters, inducible promoters, tissue-specific promoters for the desired target tissue (e.g., leaf-, root-, or flower-specific promoters when expression is desired in leaves, roots, or flowers, respectively), and whole-plant promoters. Any of these various promoters may sometimes be referred to generally as a “tissue-appropriate promoter,” Many examples of such promoters are known in the art.

Note, in particular, that a seed-specific promoter would not be considered a “tissue-appropriate promoter” within the contemplation of this invention, because expression that is specific to seeds is contrary to the purposes of this invention. By contrast, a whole-plant promoter that is also active in seeds could nevertheless be a “tissue-appropriate promoter” if it is active in leaves, roots, or flowers or other non-seed target tissues. A leaf-specific promoter would be an example of a “tissue-appropriate promoter” where leaves are the target tissues, and so forth,

We have successfully established the effectiveness of the invention in prototype demonstrations in Arabidopsis and in tobacco. Among the advantages, optional features, and preferred embodiments of the invention are the following:
1. Defending foliar tissues and other tissues of crops and other plants against bacterial and fungal pathogens.
2. Exogenous expression of seed-specific thionins in leaf tissues, root tissues, fruit tissues, flower tissues, and other non-seed tissues. Seed-specific thionins are safe for human and animal consumption.
3. Using a thionin tissue-specific signal peptide, or a signal peptide derived from a thionin tissue-specific signal peptide, to control the excretion and cleaving of thionins in target tissues, in such a manner that the thionin does not significantly damage the host cell,
4. Including in the signal peptide “Motif 3,” a 2-7 residue C-terminus sequence that contains one or more polar or acidic residues, to protect the host plant cells from lytic activity during post-translational processing and targeting to a “safe” location.
5. Including in the signal peptide “Motif 2,” a 10-14 residue hydrophobic central region.
6. Including in the signal peptide “Motif 1,” a 4-10 amino acid residue N-terminus that contains one or more basic residue(s) to stabilize excreted thionin in the plant cell walls.
7. Employing a preferred signal peptide comprising a 4-10 amino acid residue N-terminus that contains one or more basic residue(s), a 10-14 residue hydrophobic central region, and a 2-7 residue C-terminus that contains one or more acidic or polar residues.
8. Employing an exogenous gene that encodes, in addition to the signal sequence, an otherwise unmodified thionin acidic peptide domain taken from a plant species.
9. Placing the coding sequence under the control of a constitutive, inducible, general, or tissue-specific promoter—a tissue-appropriate promoter—many examples of which are known in the art.
10. Transforming crop species or other plant species with an exogenous gene in accordance with the present invention, using one of the several genetic transformation methods that are known in the art.

### BRIEF DESCRIPTION OF DRAWINGS

FIG. 1 illustrates schematically the expression cassettes in plant transformation vectors pCS35hthA, pCS35hthB, pCS35hthC, pCS35hthA-tag, pCS35hthB-tag, and pCS35hthC-tag.

FIG. 2 illustrates schematically the engineered vectors pICHthiB, pICHthiB-his, pICHthiA, and pICHthiC.

FIG. 3 depicts the signal peptide (SP), mature thionin, and I, II, and III motif sequences of several thionins.

FIG. 4 shows the relative levels of transgenic protein expression in T₀ plants, as measured by β-Glucuronidase (GUS) activity, for selected plants transformed with A: pCS35hthA; B: pCS35hthB; C: pCS35hthC; A tag: pCS35hthA-tag; pCS35hthB-tag; and C_tag: pCS35hthC-tag.

FIG. 5 shows GUS activity in T2 generation plants, indicating relative levels of αHTH transgene expression under different signal peptides.

### MODES FOR CARRYING OUT THE INVENTION

When compared in vitro to twelve well-known natural and synthetic antimicrobial peptides, including the highly potent peptides cecropin B and melittin, βPTH demonstrated the highest antifungal activity of all compounds tested (Table 1). The activity of βPTH was similar to that of the highly active antifungal antibiotics nystatin and nikkomycin Z. To verity high antibacterial and antifungal activity in vivo, βPTH and two linear antimicrobial peptides, cecropin B (moth) and phor21 (synthetic), were expressed in Arobiolopsis, under the control of the endogenous Arabidopsis chloroplast carbonic anhydrase promoter, using the rice endochitinase signal peptide for extracellular excretion of the transgenic peptides. Of the three, βPTH exhibited the greatest antibacterial and antifungal resistance, Cecropin B showed only antibacterial activity, not antifungal activity. Surprisingly, phor21 did not enhance antimicrobial resistance in vivo. In subsequent experiments, transgenic βPTH arrested fungal growth on leaf surfaces and inhibited infection of stomata, interestingly, our results suggested that the acidic C-terminal sequence of the thionin precursor was involved in folding the mature thionin. Including the acidic peptide is thus a preferred aspect of practicing the invention, although it may not be required. Tagging the βPTH precursor with EGFP greatly impaired antimicrobial activity in the mature peptide, even though EGFP was fused to the C-terminus of the acidic protein, the portion that was post-translationally cleaved.

TABLE 1
Inhibitory concentrations of antimicrobial peptides and antibiotics
tested against Rhizoctonia solani (a fungal pathogen of rice)

| | | Number of | IC₅₀ * | MIC † |
|---|---|---|---|---|
| Compound | Source | Residues | (μmol) | (μmol) |
| Cecropin B | Cecr. moth | 35 | 4.6 ± 0.3‡ | 9.8 |
| Magainin II | Afric. frog | 23 | 15.7 ± 0.9 | 30.2 |
| Melittin | Honey bee | 26 | 7.1 ± 0.6 | 13.1 |
| βPTH | Wheat | 45 | 1.14 ± 0.4 | 5.02 |
| D4E1 | Synthetic | 17 | 4.5 ± 0.3 | 8.75 |
| D2A21 | Synthetic | 23 | 6.5 ± 1.9 | 18.4 |
| pep11 | Synthetic | 10 | 11.2 ± 2.1 | 24.1 |
| phor14 | Synthetic | 14 | 19.2 ± 2.9 | 37.9 |
| phor21 | Synthetic | 21 | 8.4 ± 2.0 | 18.6 |
| Nikkomycin Z | Strept. | antibiotic | 0.86 ± 0.1 | 4.5 |
| Nystatin | Strept. | antibiotic | 1.1 ± 0.3 | 5.2 |

* IC_{50,} concentration that inhibits 50% growth of fungal cultures vs, control ± std; † MIC, minimum concentration that completely inhibits growth of fungal cells. Strept., Streptomyces. Cecr., Hyalophora cecropia. Afric. frog, Xenopus laevis

Thionins are typically 45-47 amino acids long, highly basic, and are typically active over a wide range of temperatures, even up to 60-80° C. Thionins are generally resistant to fungal proteases. The secondary structure of thionins is conserved, with a β-sheet and a double α-helix core, bound by three or four disulfide bridges. The disulfide bridges are believed to enhance the stability of the molecule, including both thermal stability and resistance to proteases. βPTH, for example, has four disulfide bonds. Crystallographic data indicate the presence of a phosphotipid-binding site in a groove formed by an arm and stem at the inner corner of the so-called Γ fold. Contributors to the phospholipid-binding site include the amino acid residues K1, S2, RIO, Y13, and R17, all of which are highly conserved among different members of the thionin family. The antifungal activity of βPTH (from wheat endosperm) was found to be significantly higher than that of either melittin or cecropin B (Table 1). Representative members of the α/β thionin family include α1- and β-purothionins from wheat seeds; α- and β-hardothionins from barley seeds; barley leaf thionins DB4, BTH6, and DG3; and oat leaf thionin Asthi1. Different thionins are often expressed in the leaves, seeds, and flowers of the same plant. Thionin genes are expressed constitutively in seeds and seedlings. Expression can be induced in leaves by methyl jasmonate or by infection with pathogenic fungi. A structural thionin gene includes regions encoding a SP, a mature thionin domain, and a C-terminal acidic protein domain. Thionins are synthesized as precursors; cleavage of both the SP and the C-terminal acidic protein yield the mature peptide.

White plant cells produce and accumulate highly lytic thionins in concentrations that are lethal to various microbial pathogens, the plant cells themselves remain largely undamaged. The mechanism underlying this differential toxicity is partially understood. In situ, the plant plasmalemma can be permeabilized by thionins, as can bacterial or fungal membranes. A C-terminal acidic protein domain may help to neutralize the basic thionin in the precursor molecule. However, after the mature thionin is cleaved from the acidic domain protein and the SP, the mature thionin should be prevented from penetrating and damaging plant membranes. Targeting and localization play a significant role in protecting plant cells. For example, the seed-specific thionins βPTH and αHTH accumulate in endosperm cells in high concentrations, and are deposited on the periphery of protein body membranes. By contrast, the leaf-specific thionins DB4 and BTH6 accumulate in the cell walls of barley leaves. Thionins are evenly distributed within the cell walls of most leaf cells in four-week-old plants. An exception was that the outer cell wall of epidermal cells was found to contain higher concentrations of thionins. High concentrations of thionins were also found in freshly formed cell-wall appositions at penetration sites following fungal infection. The transgenic oat-derived, leaf-specific thionin Asthil accumulated in cell walls when expressed in rice, similar to the behavior of barley-derived, leaf-specific thionins. However, another leaf-specific thionin from barley, DG3, was found predominantly in cell vacuoles, with less than 1% in the cell walls. An extended acidic domain appears to target the thionin DG3 to vacuoles. Also, the Si remains fused to the mature vacuolar thionin, which could explain how the protein accumulates in vacuoles without damaging host cells.

Without wishing to be bound by this hypothesis, I propose that leaf-specific SPs or other tissue-specific SPs undergo stepwise processing to control membrane permeabilization activity and cell toxicity during targeting to a “safe” destination such as the cell wall.

The binding properties of thionins may play a key role in their accumulation in plant cell walls and subsequent penetration of fungal cells. Binding to plant walls may keep thionins from inserting into the plasmalemma after the SP is cleaved and the phospholipid-binding site is activated. Thionins contain up to 10 positively charged residues that will interact electrostatically with the carboxyl groups of pectin and xylan. Various β-glucans and xylans of plant and bacterial origin bind to α1-purothionin, while cellulose and starch do not, α1-Purothionin also binds chitin, which is a principal component of fungal cell walls. Thus thionin binds to components of the primary and secondary plant cell wall, as well as to components of bacterial and fungal cell walls, Wail hydrolases, perhaps induced by phytopathogen attack, may release plant thionins and cause them to disrupt microbial membranes.

### Materials and Methods

### Examples 1-3

Plants and fungi. Arabidopsis (A. thaliana ecotype Columbia 0 (Col-0)), and all transgenic lines were grown in soil according to standard protocols. Growth and harvesting of spores from the fungus Fusarium oxysporum oxysporum f. sp. matthiolae (Dr. B. Cammue, Center of Microbial and Plant Genetics, Heverlee, Belgium) was carried out as described in Epple, P. Apel, K., and Bohlmann, H. 1997, Overexpression of an endogenous thionin enhances resistance of Arabidopsis against Fusarium oxysporum. Plant Cell 4: 509-520. Pseudomonas syringae pv tomato strain DC3000 (Dr. R. Innes, University of California, Berkeley, Calif.) was maintained as described in Whalen, M., Innes, R., Bent, A., and Staskawicz, B. 1991. Identification of Pseudomonas syringae pathogens of Arabidopsis and a bacterial locus determining avirulence on both Arabidopsis and soybean. Plant Cell 3: 49-59.

### Examples 4-9

Plant expression vectors. The wild-type αHTH precursor (Hthl, GenBank ID X05901.1) was PCR-amplified from a plasmid provided by Dr. R. Skadsen (USDA, ARS, Madison, Wis.). The αHTH precursor with a hybrid thionin SP (SPB) was obtained by fusing the αHTH precursor (corresponding to amino acids 2-138 of Hthl) with the first eleven residues of the oat Astil gene (GenBank ID AB072338.1). The αHTH precursor without the signal peptide (corresponding to residues 19-138 of Hthl) was fused by recombinant PCR to the Arabidopsis basic chitinase signal peptide (SPC) (amino acids 1-21 in Chi-B, GenBank ID NM_{—}112085), or subcloned under the rice glycine-rich protein signal peptide (SPA) (amino acids 1-27 in Grp, GenBank ID X54449). The precursor variants were cloned under the constitutive double CaMV 35S (S35) promoter (CAMBIA, Canberra, Australia) for thionin overexpression. Two sets of His₆ tag-labeted precursors were made to facilitate detection in plant tissues. The first set, S35hthA, S35hthB, and S35hthC, carried a His₆ tag at the C-terminus. The second set, S35hthA-tag, S35hthB-tag, and S35hthC-tag, carried a second His₆ tag at the N-terminus of the mature thionin, in addition to a His₆ tag at the C-terminal tag. All PCR products were verified by sequencing. All cassettes were cloned into the multiple cloning site of pCAMBIA1305.2 (CAMBIA, Canberra, Australia), and the resulting binary vectors were transferred into Agrobacterium tumefaciens strain GV3101 by electroporation.

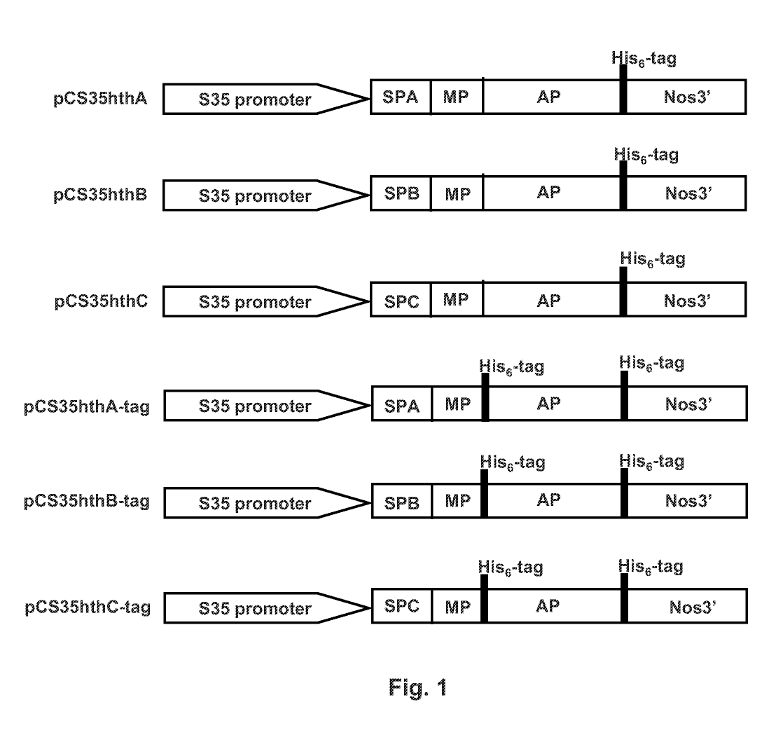

See FIG. 1, illustrating schematically the expression cassettes in plant transformation vectors pCS35hthA, pCS35hthB, pCS35hthC, pCS35hthA-tag, pCS35hthB-tag, and pCS35hthC-tag. S35=CaMV35S promoter; SPA, SPI3, and SPC=selected SPs; MP=coding region for αHTH mature peptide; AP=coding region for the acidic protein; His₆ tag=coding region for His6 tag; Nos3′=3′ untranslated termination region.

### Examples 10-15

Arabidopsis transformation and propagation. Arabidopsis Col-0 was transformed using recombinant Agrobacterium strain GV3101 by the vacuum infiltration method of Bechtold, N., and Pelletier, G. 1998. In planta Agrobacterium-mediated transformation of adult Arabidopsis thaliana plants by vacuum infiltration. Methods Mol Biol 82: 259-266. Seeds collected from the vacuum-infiltrated plants were plated in the presence of 50 mg/L hygromycin in order to select T₀ plants. At least 50 independent transformation events were analyzed for each construct, to select the 10 transformants with the highest levels of transgene expression; these plants were then used for breeding homozygous lines. The T₁ plants were allowed to self-pollinate to generate a segregating T₂ population. The 3:1 segregation of the hygromycin resistance gene was used to select single-locus transgene insertions. T₀, T1, and T₂ individuals were tested by PCR for the presence of transgenic cassettes. Primers used to identify plants transformed with pCS35hthA, pCS35hthB, pCS35hthC, and pCS35hthA-tag were the forward primers PrGRthi (5′-CCTCCTAGATCTCAAGAG-3′) (SEQ ID NO 10), PrthioB1 (5′-CTTTCCATGCGAAGTATCAAAGGTCTTAAGAGTGTAGTC-3′) (SEQ ID NO 11), PrthioC1 (5′-CTTTCCATGCGGGATCCAAGGAGATATAAC-3′) (SEQ ID NO 12), and LnT0504 (5′-GGATCCACCATCACCATCACCATTGCA-3′) (SEQ ICS NO 13), respectively; and the reverse primer PrrthioA2 (5′-CTTTCCCGGGTTAATGATGATGATGATGATGTCTAGAAAGGGATG TGAG-3′) (SEQ ID NO 14). Primers for plants transformed with pCS35hthB-tag and pCS35hthC-tag were the forward primers PrthioB1 and PrthioC1, respectively, and the reverse primer LnrT0504 (5′-ATGGTGATGGTGATGGTGGATCCTGCA-3′) (SEQ ID NO 15) for both constructs.

### Examples 16-21

RT-PCR gene expression analysis. Total RNA was extracted from leaf samples with the RNEASY Plant MiniKit (Qiagen, Valencia, Calif.). RT-PCR was performed using the One-Step RT-PCR kit (Qiagen, Valencia, Calif.). Prior to PCR, all RNA samples were treated with DNase 1 as recommended by the manufacturer. The primers were designed to amplify 370, 445, and 450-bp products, corresponding to the full-length SPA-hthl, SPB-hthl, and SPC-hthl transcripts, respectively. The same primers were used for PCR amplification of cDNAs for all constructs as above. The 375, 130, and 135-bp PCR products were designed for the transcripts SPA-hthl-tag, SPB-hthl-tag, and SPC-hthl-tag, respectively, to confirm the presence of the second tag.

### Example 22

GUS activity analysis. β-Glucuronidase (GUS) staining was performed with the fifth and sixth leaves of 5-week-old plants as otherwise described by Jefferson, R. A. 1987. Assaying chimeric genes in plants: the GUS gene fusion system. Plant Mol. Biol. Rep. 5: 387-405. An in vitro GUS assay was performed using a 4-methytumbelliferyl-β-D-glucuronide substrate. GUS activity was measured in a mixture of the seventh, eighth, and ninth leaves of 4-week-old plants, containing ˜6 μg total protein, using a Victor V multitabel counter and Walla,: 1420 Explorer software (Perkin Elmer, Boston, Mass.). To compare the GUS activity of different transgenic lines, 6-10 plants per line were assayed in two replicated experiments. Data were analyzed by one-way ANOVA with the least significant difference test, using a 95% level of significance. GUS activity was expressed in nmol 4-methylumbelliferon (MU)/(min*mg soluble proteins). Total soluble protein content was measured by the Bradford assay (Bio-Rad, Hercules, Calif.).

### Examples 23-24

Protein expression analysis. Plant tissues transformed with the recombinant vectors were examined for the presence of αHTH using Western blot analysis. Plants transformed with pCAMBIA1305.1 were used as a positive control. Leaves from four-to-five-week-old transgenic T₂ plants were homogenized in liquid nitrogen. Total cell protein was extracted with Laemmii gel loading buffer as described by Epple et al. (1997). Proteins were separated on 10-20% gradient Tricine-SDS polyacrylamide gels, and then transferred to a PVDF membrane by semi-dry electroblotting. His₆-tagged bands were detected with anti-His₆ monoclonal antibodies at 1:5000 dilution, and anti-mouse IgG horseradish peroxidase conjugate at 1:10000 dilution (BD Pharmagen), on PVDF membrane. Bound antibodies were detected with ECL Plus™ Western Blotting kit (GE Healthcare), αHTH was detected with anti-αHTH primary antibody (kindly provided by Dr. R. Skadsen) at 1:1000 dilution. Proteins were quantified by loading 100 or 200 ng of HPLC-purified αHTH, and comparing the pixel densities for 100 ng to 1 μg in the purified αHTH bands (4.9 kDa). The bands were analyzed by Kodak 1D Image Analysis Software.

### Examples 25-26

Plant resistance bioassays. Antibacterial resistance of transformants was determined by inoculating T₂ homozygous lines with the bacterial pathogen P. syringae strain DC3000. The seventh, eighth, and ninth leaves of four-week-old, soil-grown plants were syringe-injected with a bacterial suspension at a concentration of 10⁵ colony forming units (cfu)/ml as previously described by Lu (2001). Levels of bacterial growth in leaves were determined as described by Whalen et al. (1990. Each data point represented four to five replicates, with six discs per replicate. All antibacterial resistance assays were repeated twice, and analyzed by one-way ANOVA with the least significant difference test at a 95% level of significance.

Antifungal resistance was evaluated against the fungal pathogen F. oxysporum, Two-week-old T₂ progeny seedlings, grown on modified MS medium supplemented with 2% sucrose, were sprayed with a suspension of 10⁵ conidia/mi as described by Epple et al. (1997), and were then cultured for two more weeks. The plants were scored for resistance as assessed by the degree of leaf discoloration and stem browning. To assess fungal growth on leaves, seedlings were harvested and stained with trypan blue one week after inoculation as described by Keogh, R. C., Deverall, B. J., and McLeod, S. 1980,

Comparison of histological and physiological responses to Phakopsora pachyrhizi in resistant and susceptible soybean. Trand. Br. Mycol. Soc. 74: 329-333. The antifungal assays were replicated three times for each selected transgenic line.

### Examples 27-30

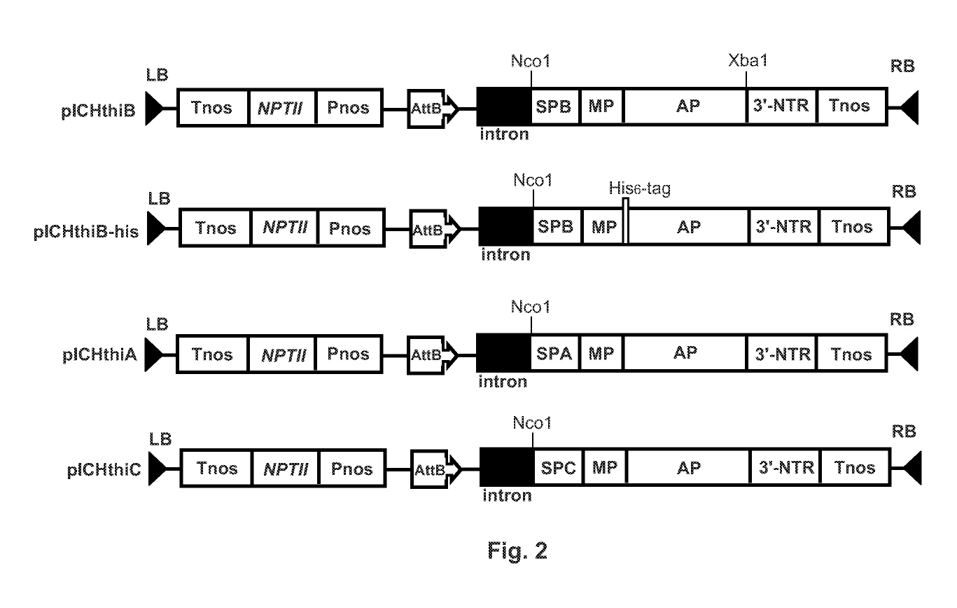

Production of thionin using recombinant plant viral-based system. The recombinant viral-based system for transient transformation of Nicotiana benthamiana, and other plant transformation vectors from Icon Genetics were as described by Marillonnet, S., Thoeringer, C., Kandzia, R., Klimyuk, V., and Gleba, Y. 2005. Systemic Agrobacterium tumefaciens-mediated transfection of viral replicons for efficient transient expression in plants. Nat Biotechnol 23: 718-723. αHTH precursor encoding regions were amplified from plasmids pCS35hthB, pCS35hthB-tag, pC S35hthA, and pC S35hthC, with the C-terminal His₆ tag excluded. The amplified sequences were cloned into the Icon Genetics vector, which encodes a 3′ module pICH11599 at NcoI-XbaI sites. FIG. 2 illustrates schematically the engineered vectors pICHthiB, pICHthiB-his, pICHthiA, and pICHthiC for expression of αHTH in N. benthamiana. Selected clones were verified by digesting with NcoI+XbaI and BamHI to distinguish between the SP fusions, and were later further verified by sequencing. The engineered vectors were transformed into Agrobacterium strain GV3101 by electroporation. Pnos=promoter; Tnos=3′ untranslated termination region; SPA, SPB, SPC=selected signal peptides; MP=coding region for the αHTH mature peptide; AP=coding region for the acidic protein; His₆ tag=coding region for His₆ tag.

### Examples 31-34

To compare the expression of αHTH under SPs of leaf and seed thionins in leaf tissues, precursors were generated by synthesizing a SP coding sequence and fusing it to the mature peptide sequence via recombinant PCR. All SP sequences were codon-optimized, to enhance expression of the thionins In particular, the αHTH precursor without the SP (corresponding to residues 19-138 of Hthl) was alternatively fused to the wheat purothionin SP (SPSd) (residues 1-27 in PurAl, GenBank ID AF004018.1), the SP of the leaf-specific barley thionin BTH6 (SPLb) (residues 1-28, GenBank ID L36882.1) (SEQ ID NO. 6), the signal peptide of the Arabidopsis leaf Thi2.1 thionin (SPLa) (residues 1-24 in Thi2.1, GenBank ID L41244.1) (SEQ ID NO. 8), or oat leaf thionin signal peptide (SPLo) (residues 1-28 in Asthil, GenBank ID AB072338.1) (SEQ ID NO. 7). The codon-optimized genes were cloned into pICH11599 as described above to generate plCHthiSd, plCHthiLb, plCHthiLa, and plCHthiLo.

The vectors were used for transformation of N. benthamiana by the protocols of Marillonnet et al. (2005). Leaves were harvested at 8 and 12 days post-transformation to test for accumulation via Western blot analysis as described above. To test the properties of transgenically expressed thionins, each thionin was extracted from leaf tissues with 0.1 N sulfuric acid and purified by the method of Jones, B., and Poulle, M. 1990. A proteinase from germinated barley: II. hydrolytic specificity of a 30 kilodalton cysteine proteinase from green malt. Plant Physiol. 94: 1062-1070. HPLC purification was performed on a C18 column (BioRad). Mass spectrometry (MS) data for exogenously generated thionins were obtained at the Mass Spectrometry Facility (LSU Department of Chemistry) and compared to those of seed-derived αHTH, which was purified from barley by the same method. The Fusarium oxysporum spore germination bioassay of Carlson, A., Skadsen, R., and Kaeppler, H. 2006. Barley hordothionin accumulates in transgenic oat seeds and purified protein retains anti-fungal properties in vitro. In Vitro Cell. Dev. Biol.—Plant 42: 318-323 was used for testing in vitro antifungal activity.

### Results

### Analysis of Signal Peptides for Transgenic Expression of Thionins.

The signal peptide directly connects to the thionin N-terminus and may have a larger effect on folding than the acidic protein at the distant C-terminus. Besides targeting the thionin to its destination, the signal peptide may also play a role in regulating post-translational processing and levels of accumulation.

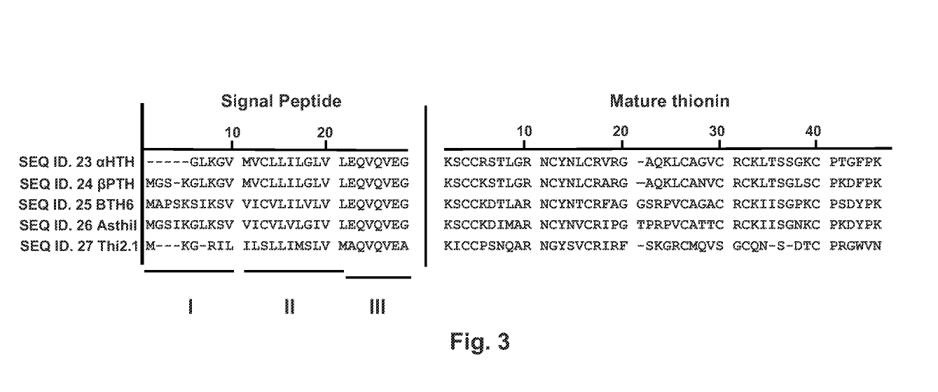

Because the membrane-permeabilizing activity of thionin is concentration-dependent, the effective inhibition of fungal growth requires a sufficient concentration of thionin in proximity to fungal membranes. We compared properties of two seed thionins, two leaf-specific thionins, and a leaf-specific γ-thionin (plant defensin) of different origins. See Table 2 and FIG. 3. Natively, barley and oat leaf-specific thionins accumulate in the cell wall, seed-specific thionins are found in protein bodies, and the Arabidopsis leaf-specific γ-thionin localization and activity in vitro are currently unknown. The barley leaf-specific thionin and the barley seed-specific thionin, BTH6 and αHTH, share only 55% homology. By contrast, the wheat seed-specific thionin and the barley seed-specific thionin, PPTH and αHTE share 85% homology.

I found that thionin SPs can have different numbers of amino acid residues and different sequences. The SPs could all be divided into three motifs, however. See FIG. 3, which depicts the signal peptide (SP), the mature thionin, and the I, II, and III motif sequences of several thionins. The SP of the barley leaf-specific thionins BD4 and BTH6, and the SP of the oat leaf-specific Asthi1 thionin each contained 28 amino acid residues. By contrast, the SPs of barley αHTH and wheat βPTH, which are found in the seed endosperm, contained 18 and 26 residues, respectively. The αHTH SP was the shortest, with only 18 residues. The SPs of βPTH and αHTH were identical, except in Motif 1. The presence of a second methionine residue in the SP of 3PTH suggested the possibility that there could, perhaps, be translation initiation at two different sites. The second initiation site should produce an 18 residue SP. Motif 3, “QVQVE,” with one acidic and two polar residues at the C-terminus of a SP, is conserved across multiple thionins, The tertiary structure of βPTH shows that the negatively charged Motif 3 is positioned in immediate proximity to the phospholipid-binding site in a precursor. This site is the most positively charged region in both thionins, and it should attract the negatively charged Motif 3. Compare S. Oard et al., “Is there a difference in metal ion-based inhibition between members of thionin family: molecular dynamics simulation study,” Biophys. Chem. 130: 65-75 (2007). Without wishing to be bound by this hypothesis, I propose that the C-terminal Motif 3 of the SP blocks the phospholipid-binding site to inhibit membrane permeabilization and toxicity, until after the thionin molecule has been delivered to its destination. When Motif 3 is absent, host plant cells can be damaged, and it is therefore strongly preferred that Motif 3 should be included in the thionin signal peptide. The hydrophobic Motif 2 was found to be typical for excreting signal peptides, containing 10-14 hydrophobic residues, and presumably adapted to span the cell membrane. The main difference between seed and leaf signal peptides was found in Motif 1. See FIG. 3. Without wishing to be bound by this hypothesis, I propose that in the case of leaf-specific thionins—and perhaps in other tissue-specific thionins as well—Motif 1 determines thionin partitioning to the cell wall, and stabilization outside the plant cell. Therefore, Motif 1 is preferably included in a signal peptide for transgenic expression of seed-specific thionins in leaf tissues.

TABLE 2
Source and sequence of thionins used for SP comparison

| | | | GenBank | | | |
|---|---|---|---|---|---|---|
| Peptide | Abr. | Source | ID | S.L.* | IC₅₀† | Transgenic expression |
| α-Hordothionin | αHTH | Barley | X05901 | Seed-PB‡ | 1 (Thevissen | Tobacco, oat (Carlson et al. |
| | | | | | et al. 1996) | 2006) |
| β-Purothionin | βPTH | Wheat | AF004018 | Seed-PB | 1.1 (Oard et | Arabidopsis (Oard and |
| | | | | | al. 2004) | Enright 2006) |
| Thionin BTH6 | BTH6 | Barley | L36882 | Leaf-CW | ND | — |
| Avenothionin 1 | Asti1 | Oat | AB072338 | Leaf-CW | ND | Rice (Iwai et at. 2002) |
| Thionin 2.1 | Thi2.1 | Arabidopsis | NM105885 | Leaf-ND | ND | Arabidopsis (Epple et al. |
| | | | | | | 1997a), tomato (Chan et al. |
| | | | | | | 2005) |

*S.L., Subcellular localization;
†IC₅₀, the concentration of a peptide that inhibits 50% growth of fungal cultures relative to control, in μmol.
‡PB, protein bodies,
CW, cell wall,
ND, not determined.

### Examples 35-37

To test these hypotheses we constructed three modified thionin precursors. To test the hypothesis that the signal peptide protects a plant cell from thionin toxicity, the signal peptide in the wild type uSITH precursor was substituted with SPC, which is the excreting signal peptide for the Arabidopsis basic chitinase. SPC has no motifs that are similar either to Motif 1 or to Motif 3, The SPC signal peptide would not be expected to protect plant cells from thionin lytic activity, and it would be expected to release the active, mature peptide outside the plasmalemma instead of stabilizing it. To test the hypothesis that a short sequence at the thionin N-terminus can block lytic activity, the wild-type signal peptide was replaced with SPA, which is the excreting signal peptide from the rice glycine rich protein. This signal peptide should place 6 extra amino acid residues at the N-terminus of a recombinant protein that would be expected to render the thionin inactive by permanently blocking the phospholipid-binding site. SPA has no Motif 1, and it would be expected to release an inactivated thionin molecule outside the plasmalemma. To test the hypothesis that Motif 1 is necessary to partition secreted thionin to the cell wall to stabilize active thionin in leaf tissues, we produced a hybrid signal peptide, SPB. Motif 1 of the oat leaf-specific thionin was fused to the wild type SP of αHTH to produce SPB (SEQ ID NO. 4). Having both Motif 1 and Motif 3, SPI3 would be expected to protect plant cells and stabilize the active thionin in the cell wall.

### Examples 38-43

Arabidopsis transformation. Six thionin precursor variants were constructed as described above and constitutively expressed in Arobidopsis. Each of the selected, hygromycin-resistant, T₀ plants generated from each cassette (as listed in FIG. 1) produced a normal phenotype. See Table 3. Stable transgene integration into the selected, hygromycin-resistant plants was confirmed by PCR analysis of genomic DNA (data not shown). Since all plasmids carried the gusP reporter gene downstream of the CaMV35S promoter, relative levels of expression in transgenic plants could be assessed by a β-giuctironidase (GUS) bioassay. First, we performed GUS staining of all hygromycin-resistant plants, followed by an in vitro assay to identify plants with strong GUS staining.

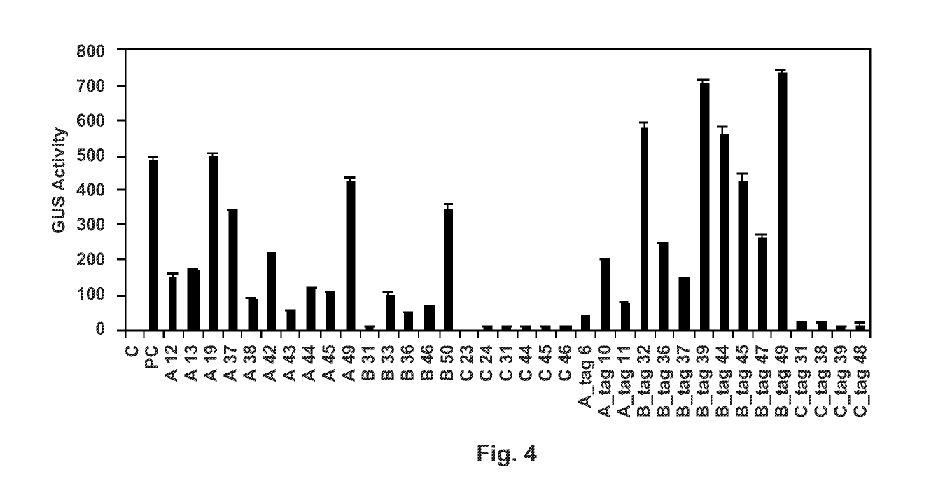

FIG. 4 shows the relative levels of transgenic protein expression in the T₀ plants, as measured by β-Glticuronidase (GUS) activity, for selected plants transformed with A: pCS35hthA; B: pCS35hthB; C: pCS35hthC; A_tag: pCS35hthA-tag; B_tag: pCS35hthB-tag; and C_tag: pCS35hthC-tag. Results shown depict the average and standard deviation of 3 replicates for each plant, per mg of fresh leaf tissue. C=untransformed Col-0 (negative control); P=the line transformed with pCA.MBIA1305.1 (positive control).

TABLE 3
Selection of transgenic T₀, T₁, and T₂ generation Arabidopsis plants

| | Number of | | | |
|---|---|---|---|---|
| | selected, | Number of T₀ | | Number of |
| | hygromycin- | plants with | Number of | selected T₂ |
| | resistant | positive | selected T₁ | homozygous |
| Vector | T₀ plants | GUS staining | sublines | lines |
| pCS35hthA | 49 | 16 | 12 | 2 |
| pCS35hthB | 50 | 19 | 13 | 2 |
| pCS35hthC | 65 | 5 | 5 | 1 |
| pCS35hthA-tag | 30 | 7 | 14 | 2 |
| pCS35hthB-tag | 52 | 14 | 9 | 2 |
| pCS35hthC-tag | 41 | 4 | 3 | 1 |

Plants transformed with plasmids encoding αHTH under the signal peptides SPA and SPE3, either with or without a His₆ tag at the N-terminus, exhibited a wide range of transgene expression. However, plants transformed with plasmids carrying the signal peptide SPC showed only low levels of transgene expression, despite additional efforts to identify plants with higher levels of expression. We screened 65 hygromycin-resistant plants transformed with pCS35hthC for a normal phenotype with little success. These results suggested that expression of αHTH with the SPC signal peptide affected Arabidopsis viability.

Expression of SPA-αHTH, SPA-αHTH-His₆ fusion, SPB-αHTH, SPA-αHTH-His₆ fusion, SPC-αHTH, and SPC-αHTH-His₆ fusion was demonstrated in transgenic plant leaves by RT-PCR of DNase-treated leaf RNA from selected T₀ plants. Gene-specific fragments of the expected size were observed in all transgenic plants, and were entirely absent from untransformed control plants.

### Examples 44-53

Independent T₁ progeny of the four to six T₀ plants with the highest GUS activity were screened for each gene cassette. Representative lines with 1:3 segregation patterns or high-level transgene expression were identified for production of T₂ homozygous progeny. To produce T2 homozygous lines, we analyzed selfed progeny of the selected T₁ lines for all six gene cassettes. For each of the best lines, three to six candidate sublines were grown and tested for: segregation, presence of the transgene, and relative levels of expression. PCR analysis of genomic DNA confirmed the presence of the full length transgenes: 933 by for the S35-SPA-hth cassette, 927 by for S35-SPB-hth, and 933 by for S35-SPC-hth. The S35-SPA-hth-tag, S35-SPA-hth-tag, and S35-SPA-hth-tag cassettes differed from the corresponding unlabeled cassettes by only six base pairs each. The presence of each of the latter was verified using 5′ primers specific for the fusion of the 5′ region of αHTH and the His₆ tag. Expression of αHTH was demonstrated in the selected homozygous T₂ lines by RT-PCR of DNase-treated leaf RNA. Gene-specific fragments of the expected size were observed in each of the T₂ generation homozygous lines HTHA13, HTHA49, HTHB1. HTHB7, HTHC31, HTHAt6, HTHAt10, HTHBt20, HTHBt39, and HTHCt31; while they were entirely absent from the untransformed control plants.

### Examples 54-59

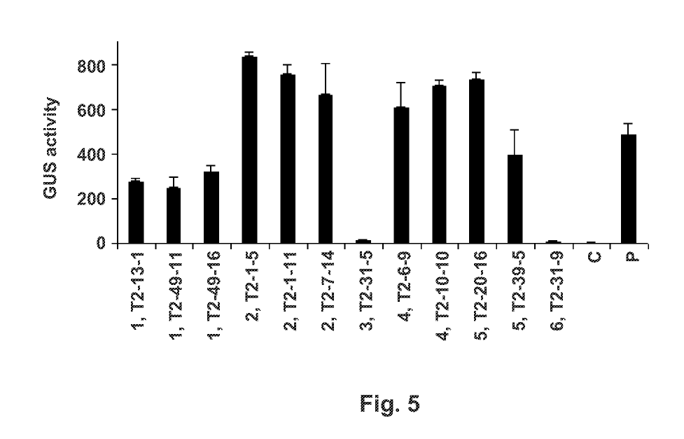

FIG. 5 shows GUS activity in T₂ plants, indicating relative levels of αHTH transgene expression under different signal peptides. Selected lines were transformed as indicated with: 1, pCS35hthA; 2, pCS35hthB; 3, pCS35hthC; pCS35hthA-tag; 5, pCS35hthB-tag; or 6, pCS35hthC-tag. These lines were named HTHA HTHB, HTHC, HTHAt, HTHBt, and HTHCt, respectively. Results shown in FIG. 5 depict the average and standard deviation of 6-8 plants, three replicates for each plant, calculated per mg of fresh leaf tissue. T2-1-1-T2-50-20=sub-lines of the independent lines; C=untransformed Col-0 (negative control); P=a line transformed with pCAMBIA1305.1 (positive control), Among the selected homozygous lines expressing unlabeled αHTH, those expressing αHTH under SPB, namely HTHB1 and HTHB7, displayed the highest GUS activity; while lines with αHTH under SPA, namely HTHA13 and HTHA49, displayed medium levels. By contrast, line PURC31, expressing αHTH under SPC, showed only a low level of activity. Among the selected lines expressing His₆ tag-labeled αHTH, the lines with αHTH under SPA and SPB showed high GUS activity; while the line HTHCt31, expressing αHTH with SPC, had low activity. These observations demonstrated the effects of the signal peptide on the viability of the transgenic Arabidopsis plants. The signal peptide SPB did not affect Arahidopsis viability, and plants expressing high levels of αHTH were detected. By contrast, the signal peptide SPC impaired the viability of the transgenic plants, and only plants with low levels of αHTH were observed.

### Examples 60-62

Immunoblot detection of αHTH in total leaf extracts from the selected homogenous lines HTHA49, HTHB1, and HTHC31 revealed differences in physical properties and accumulation of αHTH expressed with SPA, SPB, and SPC. SPB was the only signal peptide (line HTHB1) in which dimer and tetramer forms of thionin were seen, as well as the monomer. Monomer, dimer, and tetramer corresponded to 4.9, 9.7, and 19.4 kDa bands, respectively. The 4.9 kDa band of HTHB1 migrated to the same level as the HPLC-purified barley αHTH. The dimer was the most abundant species. Formation of dimers and tetramers is characteristic of wild-type thionins. These data suggest that correctly-folded αHTH was released after post-translational processing of the SPB-αHTH precursor. The main bands produced by SPA and SPC corresponded to higher molecular weights than the 4.9 kDa band of the barley αHTH. The abundant bands detected in HTHB1 showed accumulation of considerably larger amounts of the mature thionin in leaf tissues as compared to levels seen for HTHA49 or HTHC31.

### Examples 63-67

Expression under the hybrid signal peptide SPB did not affect plant viability, and produced an active αHTH (Table 4, vector pCS35hthB). The selected homozygous lines displayed enhanced resistance to F. oxvsporuin. The hybrid precursor rendered the highest antifungal resistance, up to 60%. This represented a substantial improvement over the 20% seen for untransformed plants, although it was still lower than the 80% resistance observed for the fungal-resistant Arabidopsis mutant UK-4. Although SPC impaired plant viability (vector pCS35hthC), and only low levels of transgene expression were found in plants having a normal phenotype, the SPC signal peptide nevertheless produced an active αHTH; the selected lines displayed enhanced resistance to F. oxysporum despite low levels of expression. This observation suggested that SPC did not adversely affect thionin folding. However, excretion of biologically active αHTH outside the plasmalemma under an exogenous signal sequence without Motif 1 and Motif 3 impaired plant viability, showing that the hybrid signal peptide does possess an additional function beyond merely targeting the mature peptide. The signal peptide also helped protect plant cells from thionin lytic activity during processing.

TABLE 4
Arabidopsis lines expressing αHTH with different SPs.

| | | | | | Antifungal resistance | |
|---|---|---|---|---|---|---|
| | | | The highest | | bioassay | |
| | | Number of | relative levels of | | % of infected | |
| | Signal | selected T₀ | expression* | | plants in the | |
| Vector | peptide | plants‡ | T₀ plants | T₂ lines | best T₂ line | best T₂ lines |
| pCS35hthA | SPA | 49 | 510 | 340 (±20) | 80-90 | HTHA49 |
| pCS35hthB | SPB | 50 | 420 | 810 (±15) | 40-50 | HTHB1 |
| pCS35hthC | SPC | 65 | 15 | 20 (±3) | 40-50 | HTHC31 |
| Col-0 | — | — | 0 | 0 | 80-90 | — |
| UK-4 | — | — | ND | 20-30 | — | |

‡Plants with normal phenotype.
*Relative levels of transgene expression were measured using GUS activity. Results for T₂ homozygous lines represent avg ± std.

SPA interfered with αHTH activity (vector pCS35hthA). Although several selected homozygous lines displayed relatively high levels of transgene expression, antifungal resistance remained at the level of that for untransformed plants. Based on the results seen for vector pCS35hthC, it is believed that this inactivity was presumably not caused by misfolding. Rather, the explanation could lie in the presence of the extra amino acid residues, blocking the phospholipid-binding site. These results support the hypothesis that the C-terminal motif of the thionin SP plays an important role in protecting plant cells, by blocking the activity of the mature peptide during targeting to a “safe” destination such as the cell wall. This function evidently belongs to Motif 3.

Abundant accumulation of the mature thionin in leaf tissues was observed only with SPB, which alone contained Motif 1. Excretion of the inactive thionin, as in the case of SPA, did not increase accumulation in leaf tissues. Excretion of the biologically active and correctly folded mature thionin, as in the case of SPC, did not suffice to cause the accumulation of thionin in leaf tissues. These data indicated that Motif 1 plays an important role in stabilizing excreted thionin in leaf tissues.

### Examples 68-75

Production of αHTH with a Recombinant Plant Viral-Based System, to Test the Effects of the Signal Peptides.

To further investigate the effects of the signal peptide sequence on folding, stability, localization, and activity of seed-specific thionins in leaf tissues, αHTH precursor variants were cloned and transiently expressed in N. benthamiana. This expression system allows one to produce milligram quantities of protein, amounts that suffice to purify and characterize peptides (See Marillonnet et at. 2005). We used the same signal peptides as in the Arabidopsis experiment, SPA, SPB, and SPC. In addition, four native thionin signal peptides were placed in front of the αHTH coding sequence. See Table 5. Eight days after the transformations, leaves were harvested and αHTH was measured in total protein extracts. Barky seedling αHTH was used as a positive control, Each variant produced a band ˜4,9 kDa (or larger), corresponding to αHTH. Identity was confirmed by Western blot analysis using an anti-αHTH primary antibody, Extraction with 0.1 N sulfuric acid followed by HPLC purification according to the protocol of Jones et at. (1990) yielded good amounts of recombinant peptide.

TABLE 5
Expression of αHTH in N. benthamiana

| Plant | Vector | Signal peptide | Yield* |
|---|---|---|---|
| barley† | — | — | 30 |
| Nicotiana | pICHthiA | SPA, Rice glycine rich protein | 2300 |
| Nicotiana | pICHthiB | SPB, Hybrid αLITH | 576 |
| Nicotiana | pICHthiC | SPC, Arabidopsis basic chitinase | 580 |
| Nicotiana | pICHthiD | SPD, Apple p48978 apoplast | 63 |
| Nicotiana | pICHthiE | SPE, Calreticulin apoplast | 54 |
| Nicotiana | pICHthiSd | SPSd, wheat purothionin (seed-specific) | ND |
| Nicotiana | pICHthiLb | SPLb, barley leaf-specific thionin BTH6 | ND |
| Nicotiana | pICHthiLa | SPLa, Arabidopsis leaf thionin Thi2.l | ND |
| Nicotiana | pICHthiLo | SPLo, oat leaf-specific thionin Asthi1 | ND |

*Yield in μg/g fresh weight; †leaf thionin.

The HPLC patterns of extracts of transgenic WITH expressed under different signal peptides displayed variation in post-translational processing. MS analysis of the major fractions revealed that only SPB released the correctly processed mature peptide, with a molecular weight corresponding to 45 amino acid residues (Table 6). No additional peaks were found for SPB, indicating the prevalent accumulation of the correctly-folded mature peptide. By contrast, SPA released a 47-residue peptide with two extra residues at the N-terminus, A minor peak corresponding to a 48-residue product was also found for SPA. The main product for SPC carried one extra residue at the N-terminus, a glutamic acid, indicating incorrect processing. A minor, 43-residue peak for SPC pointed to truncation and reduced stability.

SPB produced three additional HPLC fractions that eluted before the major one, while SPC and SPA produced two and none, respectively. MS analysis confirmed that these preceding fractions mainly contained truncated versions of the mature peptides. The data indicated directed cleavage of the mature thionin in the cases of SPB and SPC, unlike the case for SPA. Unlike SPB, extracts for SPA and SPC contained relatively large fractions that eluted immediately after the major peak. The SPA and SPC fractions contained mainly proteins with the same molecular weight as that for the major peak, indicating misfolding.

TABLE 6
MS analysis of αHTH extracts purified from Nicoliana benthamiana
leaves expressing αHTH with different SPs.

| | | Molecular weight (kDa) | | |
|---|---|---|---|---|
| | | | Additional | |
| | Signal | Main | peaks in | Main peaks in |
| Vector | peptide | fraction | main fraction | additional fractions |
| pICHthiaA | SPA | 5075 (47)^{a} | 5188 (48) | 5075 (47), 5188 (48), |
| | | | | 5955 (?) 3797 (?), |
| | | | | 4475 (42), 4975 (46), |
| pICHthiaB | SPB | 4847 (45) | None* | 5069 (47), 5982 (55?), |
| pICHthiaC | SPC | 4976 (46) | 4604 (43) | ND |

*Concentrations below detectable levels. ^{a}Number of amino acid residues corresponding to the determined molecular weight is shown in parentheses.

### Example 76

The novel thionin expression strategy disclosed here may he used to enhance resistance to pathogens in many crops and ornamental plant species, including for example rice, maize, soybean, sorghum, millet, and roses. As just one example, it may be used in flower tissues in maize to inhibit Aspergillus infections that can lead to aflatoxin. After resistant lines are obtained through transgenic methods, those lines may be crossed and backcrossed with local varieties using breeding techniques well known in the art to develop resistant varieties and hybrids that are adapted to local conditions in various countries, and that have agronomically desirable characteristics.

### Example 77

Thionins are part of the plant innate immune system. As such, thionins undergo accelerated evolution under continuous selective pressure from pathogenic microorganisms. I have explored the Hordeum vulgare genome, and found many, many homologues of seed thionins (hordothionins) within this single genome. Nearly fifty homologues of αHTH were identified in the Hordeum vulgare genome. The barley genome project is ongoing; however, a partially completed Hordeum vulgare genome, HvGDB, is publicly available at http://www.plantgdb.org/HvGDB/. To identify αHTH homologues in the Hordeum vulgare genome, the αHTH precursor amino acid sequence (GenBank ID: CAA29330.1) was queried against HvGDB using BLAST software, using the tblastn option (to search a nucleotide database using a protein query). In particular, the PlantGDB BLAST was used with the following options: Barley1 GeneChip Exemplars database and PUT (contigs assembled from EST and cDNA) of Hordeum vulgare (based on GenBank release 169). When the mature sequence was queried, our search identified nearly fifty homologues with BLAST E-values ranging from 2×10⁻²⁰ to 2×10⁻⁹, corresponding to 100% to 66% homology, respectively. Of note, all the conserved motifs for the mature thionin domain were found in each of the identified homologues. Similar genomic searches may be used to identify other seed-derived thionins, in the same or other species. Any of these seed-derived thionins may be used in practicing the present invention.

Cloning into Other Green Plants.

The novel disease resistance nucleotide sequences may be used to transform disease resistance into green plants generally. Resistance may be then introduced into other allospecific or conspecific plants, for example, either by traditional breeding, back-crossing, and selection; or by transforming cultivars with the cloned nucleotide sequences. Direct transformation of cultivars has the potential to allow quick introduction of the resistance characteristics into a variety, without requiring multiple generations of breeding and back-crossing to attain uniformity.

It will be understood by those skilled in the art that the listed nucleic acid sequences are not the only sequences that can be used to confer antimicrobial and antifungal resistance. Also contemplated are those nucleic acid sequences that encode identical proteins or peptides but that, because of the degeneracy of the genetic code, possess different nucleotide sequences. For example, it is well known in the art that the codon for asparagine may be either AAT (AAU) or AAC.

The invention also encompasses nucleotide sequences encoding peptides or proteins having one or more silent amino acid changes in portions of the molecule not directly involved with antimicrobial properties. For example, alterations in the nucleotide sequence that result in the production of a chemically equivalent amino acid at a given site are contemplated; thus, a codon for the amino acid alanine, a hydrophobic amino acid, may be substituted by a codon encoding another hydrophobic residue, such as glycine, or may be substituted with a more hydrophobic residue such as valine, leucine, or isoleucine. Similarly, changes that result in the substitution of one negatively-charged residue for another, such as aspartic acid for glutamic acid, or one positively-charged residue for another, such as lysine for arginine, can also be expected to produce a biologically equivalent product.

This invention relates not only to a functional thionin and signal peptide sequence as described in this specification, but also to peptides having modifications to such a sequence resulting in an amino acid sequence having the same function (i.e., a functional thionin with antimicrobial or antifungal activity, not injurious to the host cell, excreted and associated with the cell wall in leaves), and about 60-70%, preferably 90% or greater homology to the sequence of the amino acid sequence as described, more preferably about 95% or greater homology, particularly in conserved regions. “Homology” as used here means identical amino acids or conservative substitutions (e.g., acidic fur acidic, basic for basic, polar for polar, nonpolar for nonpolar, aromatic for aromatic). The degree of homology can be determined by simple alignment based on programs known in the art, such as, for example. GAP and PILEUP by GCG, or the BLAST software available through the NIH internet site. Most preferably, a certain percentage of “homology” would be that percentage of identical amino acids.

A particular desired point mutation may be introduced into a coding sequence using site-directed mutagenesis methods known in the art. Isolated DNA sequences of the present invention are useful to transform target crop plants or ornamental, and thereby confer antimicrobial or antifungal resistance. A broad range of techniques currently exists for achieving the direct or indirect transformation of higher plants with exogenous DNA, and any method by which one of the novel sequences can be incorporated into the host genome, and stably inherited by its progeny, is contemplated by the present invention.

Transformation of plant cells can be mediated by the use of vectors. A common method for transforming plants is the use of Agrobacterium tumefaciens to introduce a foreign nucleotide sequence into the target plant cell. For example, a thionin nucleotide sequence is inserted into a plasmid vector containing the flanking sequences in the Ti-plasmid T-DNA. The plasmid is then transformed into E. coli. A triparental mating is carried out among this strain, an Agrobacterium strain containing a disarmed Ti-plasmid containing the virulence functions needed to effect transfer of the thionin-containing I-DNA sequences into the target plant chromosome, and a second E. coli strain containing a plasmid. having sequences necessary to mobilize transfer of the thionin construct from E. coli to Agrobacterium. A recombinant Agrobacterium strain, containing the necessary sequences for plant transformation, is used to infect leaf discs. Discs are grown on selection media and successfully transformed regenerants are identified.

Plant viruses also provide a possible means for transfer of exogenous DNA.

Direct uptake of DNA by plant cells can also be used. Typically, protoplasts of the target plant are placed in culture in the presence of the DNA to be transferred, along with an agent that promotes the uptake of DNA by protoplasts. Such agents include, for example, polyethylene glycol and calcium phosphate.

Alternatively, DNA uptake can be stimulated by electroporation. In this method, an electrical pulse is used to open temporary pores in a protoplast cell membrane, and DNA. in the surrounding solution is then drawn into the cell through the pores. Similarly, microinjection can be used to deliver the DNA directly into a cell, preferably directly into the nucleus of the cell.

In many of these techniques, transformation occurs in a plant cell in culture. Subsequent to the transformation event, plant cells must be regenerated to whole plants. Techniques for the regeneration of mature plants from callus or protoplast culture are known for a large number of plant species. See, e.g., Handbook of Plant Cell Culture, Vols. 1-5, 1983-1989 McMillan, N.Y.

Alternate methods are also available that do not necessarily require the use of isolated cells and plant regeneration techniques to achieve transformation. These are generally referred to as “ballistic” or “particle acceleration” methods, in which DNA-coated metal particles are propelled into plant cells by either a gunpowder charge (see Klein et al., Nature 327: 70-73, 1987) or by electrical discharge (see EPO 270 356). In this manner, plant cells in culture or plant reproductive organs or cells, e.g. pollen, can be stably transformed with the DNA sequence of interest,

in certain dicots and monocots, direct uptake of DNA is the preferred method of transformation. For example, in maize or rice the cell wall of cultured cells is digested in a buffer with one or more cell wall-degrading enzymes, such as cellulose, hemiceilulase, and pectinase, to isolate viable protoplasts. The protoplasts are washed several times to remove the degrading enzymes, and are then mixed with a plasmid vector containing the nucleotide sequence of interest, The cells can be transformed with either PEG (e.g. 20% PEG 4000) or by electroporation. The protoplasts are placed on a nitrocellulose filter and cultured on a medium with embedded maize cells functioning as feeder cultures. After 2-4 weeks, the cultures in the nitrocellulose alter are maintained in medium for 1-2 months. The nitrocellulose filters with the plant cells are transferred to fresh medium nurse cells every two weeks. Optionally, selective pressure may be applied by inoculating the medium with pathogenic bacteria or pathogenic fungi to which the plant cells would normally be susceptible, but against which the thionin provides protection. The un-transformed cells cease growing and die after a time in response to this selective pressure.

Other methods of transforming plants are described in B. Jenes et al., and in S. Ritchie et al., in S. -D. Kung et al. (Eds.), Transgenic Plants, vol. 1, Engineering and Utilization, Academic Press, Inc., Harcourt Brace Jovanovich (1993); and in L. Marmonen et al., Critical Reviews in Biotechnology, vol. 14, pp. 287-310 (1994). See also the various references cited on pages 15-17 of published international patent application WO 00/26390, each of which is incorporated by reference.

A particularly preferred transformation vector, which may be used to transform seeds, germ cells, whole plants, or somatic cells of monocots or dicots, is the transposon-based vector disclosed in U.S. Pat. No. 5,719,055. This vector may be delivered to plant cells through one of the techniques described above or, for example, via liposomes that fuse with the membranes of plant cell protoplasts.

The present invention can be applied to transform virtually any type of green plant, both monocot and dicot. Among the crop plants and other plants for which transformation is contemplated are (for example) rice, maize, wheat, millet, rye, oat, barley, sorghum, sunflower, sweet potato, cassava, alfalfa, sugar cane, sugar beet, canoia and other Brassica species, sunflower, tomato, pepper, soybean, tobacco, melon, lettuce, celery, eggplant, carrot, squash, melon, cucumber and other cucurbits, beans, cabbage and other eruciferous vegetables, potato, tomato, peanut, pea, other vegetables, cotton, clover, cacao, grape, citrus, strawberries and other berries, fruit trees, and nut trees. The novel sequences may also be used to transform turf grass, ornamental species, such as petunia and rose, and woody species, such as pine and poplar.

### Miscellaneous

Through routine breeding practices known in the art, progeny will be bred from successfully-transformed parent plants. Once progeny are identified that are demonstrably resistant to bacterial or fungal infection, those progeny will be used to breed varieties and hybrids for commercial use. Crossing and back-crossing resistant plants with other germplasm through standard means will yield thionin-expressing varieties and hybrids having good productivity and other agronomically desirable properties. Alternatively, direct transformation into a variety or into a parent of a hybrid having agronomically desirable properties may be employed, as direct transformation can accelerate the overall selection and breeding process.

As used in the specification and claims, unless otherwise clearly indicated by context, the term “plant” is intended to encompass plants at any stage of maturity, as well as any cells, tissues, or organs taken or derived from any such plant, including without limitation any embryos, seeds, leaves, stems, flowers, fruits, roots, tubers, single gametes, anther cultures, callus cultures, suspension cultures, other tissue cultures, or protoplasts. Also, unless otherwise clearly indicated by context, the term “plant” is intended to refer to a photosynthetic organism or green plant including algae, mosses, ferns, gymnosperms, and angiosperms. The term excludes, however, both prokaryotes, and eukaryotes that do not carry out photosynthesis such as yeast, other fungi, and the so-called red plants and brown plants that do not carry out photosynthesis.

Unless otherwise clearly indicated by context, the “genome” of a plant refers to the entire DNA sequence content of the plant, including nuclear chromosomes, mitochondrial chromosomes, chloroplast chromosomes, plasmids, and other extra-nuclear or extra-chromosomal DNA,

Unless otherwise clearly indicated by context, the “progeny” of a plant includes a plant of any subsequent generation whose ancestry can be traced to that plant.

Unless otherwise clearly indicated by context, a “derivative” of a thionin transformed plant includes both the progeny of that plant, as the term “progeny” is defined above; and also any mutant, recombinant, or genetically-engineered derivative of that plant, whether of the same species or of a different species; where, in either case, the thionin defensive peptide characteristics of the original plant have been transferred to the derivative plant. Thus a “derivative” of a plant could include, by way of example and not limitation, any of the following plants that express the same thionin defensive peptide: F₁ progeny plants, F₂ progeny plants, F₃₀ progeny plants, a transgenic maize plant transformed with a thionin defensive peptide derived from barley, and a transgenic sweet potato plant so transformed.

The following definitions should be understood to apply throughout the specification and claims, unless otherwise clearly indicated by context.

An “isolated” nucleic acid sequence is an oligonucleotide sequence that is located outside a living cell. A cell comprising an “isolated” nucleic acid sequence is a cell that has been transformed with a nucleic acid sequence that at one time was located outside a living cell; or a cell that is the progeny of, or a derivative of, such a cell.

In one embodiment, the invention comprises a polynucleotide adapted to cause the expression of a thionin in a target plant tissue; wherein: (a) the polynucleotide comprises a promoter and a coding sequence, wherein the promoter is operatively linked to the coding sequence; (b) the promoter is a tissue-appropriate promoter for a target plant tissue or tissues, wherein the target plant tissue or tissues are selected from the group consisting of leaf tissue, root tissue, flower tissue, and fruit tissue; (c) the coding sequence encodes a peptide comprising a signal peptide domain and a thionin domain; (d) the thionin domain is identical to a native thionin from a seed from a plant species, or the thionin domain has 80%, 85%, 90%, 95%, or 100% homology to the amino acid sequence of a native thionin from a seed from a plant species; (e) the signal peptide is adapted to cause the excretion of the thionin domain from a plant cell, if the polynucleotide should be transcribed and translated in a plant cell; and the signal peptide comprises three motifs: a C-terminal motif, an excretion motif, and an N-terminal motif (f) the C-terminal motif consists of from 2 to 7 amino acid residues, comprising one or more acidic amino acid residues, and containing no basic amino acid residues; and wherein, if the polynucleotide should be transcribed and translated in a plant cell, then the C-terminal motif will block the lytic activity of the expressed thionin during transport. of the thionin within the cell; (g) the excretion motif comprises from 10 to 14 nonpolar amino acid residues; the excretion motif is identical to a native excretion motif from a plant signal peptide; and wherein, if the polynucleotide should be transcribed and translated in a plant cell, then the excretion motif is adapted to span the membrane of the plant cell, and thereby to promote the excretion of the thionin through the membrane; (h) and the N-terminal motif comprises from 4 to 10 amino acid residues, comprising one or more basic amino acid residues; the N-terminal motif is identical to an N-terminal motif from a plant thionin signal peptide that is specific for the same target plant tissue and that is native to the same plant species, or that is specific for the same target plant tissue and that is native to a different plant species; and wherein, if the polynucleotide should be transcribed and translated in a plant cell, then the N-terminal motif is adapted to stabilize the excreted thionin in the plant cell wall, or to inhibit reinsertion of the excreted thionin into the plasmalemma, or both.

In other embodiments: (a) The coding sequence encodes a peptide comprising a signal peptide domain, a thionin domain, and a C-terminal acidic peptide domain; and the acidic peptide domain is identical to a native acidic peptide domain associated with a thionin from a plant species, or the acidic peptide domain has 80%, 85%, 90%, 95%, or 100% homology to a native acidic peptide domain associated with a thionin from a plant species. Or (b) The C-terminal motif is identical to a native C-terminal motif from a thionin signal peptide from a plant species. Or (c) The thionin domain is identical to a. native thionin from a seed from a plant species, but with one or two additional amino acid residues on the N-terminus of the thionin domain as compared to the native thionin. Or (d) The polynucleotide is an isolated, recombinant, mutagenized, or synthetic polynucleotide.

Other embodiments include: (a) A transformation vector comprising the polynucleotide. Or (b) A host cell comprising the polynucleotide, Or (c) A method for producing a plant having enhanced resistance to funigal infection, comprising transforming plant cells with the polynucleotide, wherein the plants cells are capable of regenerating a plant. Or (d) A plant produced by such a method, wherein cells of the plant express the encoded thionin. Or (e) A derivative plant of such a plant, wherein cells of the derivative plant express the encoded thionin. Or (f) A seed of such a plant or derivative plant, or capable of producing such a derivative plant, wherein cells of the seed comprise the polynucleotide.

Other embodiments include: (a) A method for producing a plant having enhanced resistance to fungal infection, the method comprising crossing or back-crossing such a plant or derivative plant with other germplasm to produce a progeny plant, wherein cells of the progeny plant express the encoded thionin. Or (b) A plant produced by such crossing or backcrossing, wherein cells of the plant express the encoded thionin, Or (c) A derivative of such a plant, wherein cells of the derivative plant express the encoded thionin. Or (d) A seed of such a plant or derivative plant, wherein cells of the seed comprise the polynucleotide.

Other embodiments include such a plant or derivative plant, wherein the plant is a monocot, or wherein the plant is a dicot.

The complete disclosures of all references cited in the specification are hereby incorporated by reference, including the complete disclosures of the references listed in the following bibliography, and the complete disclosure of the priority application, U.S. provisional patent application Ser. No. 61/313,458, filed 12 Mar. 2010. In the event of an otherwise irreconcilable conflict, however, the present specification shall control,

### REFERENCES

D. Florack et al., “Expression of biologically active hordothionins in tobacco. Effects of pre- and pro-sequences at the amino and carboxyl termini of the hordothionin precursor on mature protein expression and sorting,” Plant Molecular Biology, vol. 24, pp. 83-96 (1994)

S. Oard et al., “Structural changes induced in thionins by chloride anions as determined by molecular dynamics simulations,” Biophysical Chemistry, vol. 147, pp. 42-52 (2010)

S. Oard et al., “Tuning exogenous expression of a wheat antimicrobial peptide purothionin,” Keystone Symposium on Molecular and Cellular Biology “Plant Innate Immunity”. Keystone, Colo., February 2008, p. 128,

Agrios, G. 1997. Plant Pathology, Fourth Ed. ed. Academic Press, London, UK, Bechtold, N., and Pelletier, G. 1998. In planta Agrobacterium-mediated transformation of adult Arabidopsis thaliana plants by vacuum infiltration. Methods Mot Riot 82: 259-266,

Bohlmann, H., Clausen, S., Behnke, S., Giese, H., Hiller, C., Reimann-Philipp, U., Schrader, G., Barkholt, V., and Apel. K. 1988. Leaf-specific thionins of barley—a novel class of cell wall proteins toxic to plant-pathogenic fungi and possibly involved in the defense mechanism of plants. EMBO J 7: 1559-1565.

Bourne, J. J. 2009. The Global Food Crisis: The End of Plenty. National Geographic.

Brett, C., and Waldron, K. 1996. Physiology and Biochemistry of Plant Cell Walls, 2 ed. Chapman and Hall, London.

Brümmer, J., Thole, H., and Kloppstech, K. 1994. Hordothionins inhibit protein synthesis at the level of initiation in the wheat-germ system. Eur Biochem 219: 425-433.

Caaveiro, J., Molina, A., Rodriguez-Paienzuela, P., Goni, F., and Gonzalez-Manas, J. 1998. Interaction of wheat {alpha}-thionin with large unilameliar vesicles. Protein Sci. 7: 2567-2577.

Carlson, A., Skadsen, R., and Kaeppler, H. 2006. Barley hordothionin accumulates in transgenic oat seeds and purified protein retains anti-fungal properties in vitro. In Vitro Cell, Dcv. Biol.—Plant 42: 318-323.

Carmona, M., Hernadez-Lucas, C., San Martin, C., Gonzalez, P., and Garcia-Ohnedo, F. 1993a. Subcellular localization of type 1 thionins in the endosperms of wheat and barley. Protoplasma 173: 1-7.

Carmona, M., Molina, A., Fernández, J., López-Fando, J., and Garcia-Olmedo, F. 1993b. Expression of the alpha-thionin gene from barley in tobacco confers enhanced resistance to bacterial pathogens. The Plant Journal 3: 457-462,

Chan, Y., Prasad, V., Chen, K., Liu, P., Chan, M., and Cheng, C. 2005. Transgenic tomato plants expressing an Arabidopsis thionin (Thi2.1) driven by fruit-inactive promoter battle against phytopathogenic attack. Planta 221: 386-393.

Dubreila, L., Gaborita, I., Bouchetc, B., Gallantc, D. J., Broekaertd, Quilliena, L., and Marion, D. 1998. Spatial and temporal distribution of the major isoforms of puroindohnes (puroindotine-a and puroindoline-b) and non specific lipid transfer protein (ns-LTP1e1) of Triticum aestivum seeds., Plant Science 138: 121-135

Ebrahim-Nesbat, F., Behnke, S., Kleinhofs, A., and Apel, K. 1989. Cultivar-related differences in the distribution of cell-wall-bound thionins in compatible and incompatible interactions between barley and powdery mildew Planta 179: 203-210.

Epple, P., Apel, K., and Bohlmann, H. 1997. Overexpression of an endogenous thionin enhances resistance of Arabidopsis against Fusarium oxysporum. Plant Cell 4: 509-520.

Epple, P., Vignutelli, A., Apel, K., and Bohlmann, H. 1998. Differential induction of the Arabidopsis thaliana Thi2.1 gene by Fusarium oxysporum f. sp. matthiolae. Molecular Plant-Microbe Interactions 11: 523-529,

Evans, J., Wang, Y., Shaw, K., and Vernon, L. 1989. Cellular responses to Pyrularia thionin are mediated by Ca2+ influx and phospholipase A2 activation and are inhibited by thionin tyrosine iodination. Proc. Nail Acad Sci USA 86: 5849-5853.

Florack, D., and Stiekema, W. 1994. Thionins: properties, possible biological roles and mechanisms of action. Plant Mol. Biol. 26: 25-37.

Fracki, W., Li, D., Owen, N., Perry, C., Naisbitt, G., and Vernon, L. 1992. Role of Tyr and Trp in membrane responses of Pyrularia thionin determined by optical and NMR spectra following Tyr iodination and Trp modification. Toxicon 30: 1427-1440.

Garcia-Olmedo, F., Molina, A., Alamillo, J. M., and Rodriguez-Palenzueta. P. 1998. Plant defense peptides. Biopolymers 47: 479-491.

Garcia-Olmedo, F., Rodriguez-Palenzueta, P., Hernandez-Lucas, C., Ponz, F., Maraña, C., Carmona, M., Lopez-Fando, J., Fernandez, J., and Carbonero, P. 1989. The thionins: a protein family that includes purothionins, viscotoxins and crambins. Oxford Surveys Plant Mol. Cell Biol. 6: 31-60.

Hughes, P., Dennis, E., Whitecross, M., Llewellyn, D., and Gage, P. 2000. The cytotoxic plant protein, b-purothionin, forms ion channels in lipid membranes. J. Biol. Chem. 275: 823-927.

Iwai, T., Kaku, H., Honkura, R., Nakamura, S., Ochiai, H., Sasaki, T., and Ohashi, Y, 2002. Enhanced resistance to seed-transmitted bacterial diseases in transgenic rice plants overproducing an oat cell-wall-bound thionin Mol Plant Microbe Interact 15: 515-521,

Jefferson, R. A. 1987. Assaying chimeric genes in plants: the GUS gene fusion system. Plant Mol. Biol. Rep. 5: 387-405.

Jones, B., and Mak, A. 1977. Amino acid sequences of the two α-purothionins of hexaploid wheat. Cereal Chemistry 54: 511-523.

Jones, B., and Poulle, M. 1990. A proteinase from germinated barley: 11. hydrolytic specificity of a 30 kilodalton cysteine proteinase from green malt. Plant Physiol. 94: 1062-1070.

Joshi, B. N., Sainani, M., Bastawade, K., Gupta, V., and Ranjekar, P. K. 1998. Cysteine protease inhibitor from pearl millet: a new class of antifungal protein. Biochemical and Biophysical Research Communications 246: 382-387.

Keogh, R. C., Deyerail, B. J., and McLeod, S. 1980. Comparison of histological and physiological responses to Phakopsora pachyrhizi in resistant and susceptible soybean. Trand. Br. Mycol. Soc. 74: 329-333.

Kitanaga, Y., Jian, C., Hasegawa, Ni., Yazaki, J., Kishimoto, N., Kikuchi, S., Nakamura, H., Ichikawa, H., Asami, T., Yoshida, S., et al, 2006. Sequential regulation of gibberellin, brassinosteroid, and jasmonic acid biosynthesis occurs in rice coleoptiles to control the transcript levels of anti-microbial thionin genes. Biosci. Biotech. Biochem. 70: 2410-2419.

Mak, A., and Jones, B.1976. Separation and characterisation of chymotryptic peptides from alpha- and beta-purothionins of wheat. J. Sci. Food Agr. 27: 205-213,

Maillonnet, S., Thoeringer, C., Kandzia, R., Klitnyuk, V and Gleba, Y. 2005. Systemic Agrobacterium tumefaciens-mediated transfection of viral replicons for efficient transient expression in plants. Nat Biotechnol 23: 718-723.

Molina, A., Segura, A., and Garcia-Ohnedo, F. 1993. Lipid transfer proteins (nsLTPs) from barley and maize leaves are potent inhibitors of bacterial and fungal plant pathogens. FEBS Lett. 316: 119-122.

Oard, S., and Enright, F. 2006. Expression of the antimicrobial peptides in plants to control phytopathogenic bacteria and fungi. Plan Cell Rep. 25: 561-572.

Oard, S., Karki, B., and Enright, F. 2007. Is there a difference in metal ion-based inhibition between members of thionin family: molecular dynamics simulation study. Biophys. Chem. 130: 65-75,

Oard, S., and Karki, B. B. 2006, Mechanism of β-purothionin antimicrobial peptide inhibition by metal ions: molecular dynamics simulation study. Biophys. Chem. 121: 30-43.

Oard, S., Rush, M. C., and Oard, J. H. 2004. Characterization of antimicrobial peptides against a US strain of the rice pathogen Rhizoctonia solani. J. Appl. Microbiol. 97: 169-180,

Oita, S., Ohnishi-Kameyama, M., and Nagata. T. 2000. Binding of barley and wheat alpha-thionins to polysaccharides. Biosci. Biotech. Biochem. 64: 958-964.

Pekkarinen, A., and Jones, B. 2003. Purification and identification of barley (Hordeum vulgare L.) proteins that inhibit the alkaline serine proteinases of Fusarium culmorum. J Agric Food Chem 51: 1710-1717.

Pekkarinen, A., Jones, B., and Niku-Paavola. M. 2002. Purification and properties of an alkaline proteinase of Fusarium culmorum. Eur J Biochem 269: 798-807.

Rao, U., Stec, B., and Teeter. M. 1995. Refinement of purothionins reveals solute particles important for latice formation and toxicity. 1. al-purothionin revisited. Acta Cystallogr., Sect. D D51: 904-913,

Ravapuram, C., Wu, J., Haas, C., and Baldwin, 1. 2008. PR-13/Thionin but not PR-1 mediates bacterial resistance in Nicotiana attenuata in nature, and neither influences herbivore resistance. Molecular Plant-Microbe Interactions 21: 988-1000.

Redman, D. G. and Fisher, N. 1969. Purothionin analogues from barley flour. J. Sci. Food Agr. 20: 427-432.

Reimann-Philipp, U., Behnke, S., Batschauer, A., Schäfer, E., and Apel, K. 1989. The effect of light on the biosynthesis of leaf-specific thionins in barley, Hordeum vulgare. Eur. J. Biochem. 182: 283-289.

Richard, J. -A., Kelly, I., Marion, D., Pezolet, M., and Auger, M. 2002. Interaction between b-purothionin and dimyristoylphosphatidyiglycerol: A ³¹P-NMR and infrared spectroscopic study. Biophys. J. 83: 2074-2083.

Richard, J., Kelly, I., Marion, D., Auger, M., and Pézolet, M. 2005 Structure of beta-purothionin in membranes: a two-dimensional infrared correlation spectroscopy study. Biochemistry 44: 52-61.

Romero, A., Alamillo, J., and Garcia-Oirnedo, F. 1997. Processing of thionin precursors in barley leaves by a vacuolar proteinase. Eur J Biochem 243: 202-208.

Sels, J., Mathys, J., De Coninck, B., Cammue, B., and De Bone, M. 2008. Plant pathogenesis-related (PR) proteins: a focus on PR peptides. Plant Physiol. Biochem. 46: 941-950.

Sreedhar, L., Kobayashi, D., Bunting, T., Hillman, B., and Belanger, F. 1999. Fungal proteinase expression in the interaction of the plant pathogen Magnaporthe poae with its host, Gene 235: 121-129.

Stec, B., Markman, O., Rao, U., Heffron, G., Henderson, S., Vernon, L. P., Brumfeld, V., and Teeter, M. M. 2004. Proposal for molecular mechanism of thionins deduced from physicochemical studies of plant toxins. J. Pep. Res. 64: 210-224.

Stec, B., Rao, U., and Teeter, M. M. 1995. Refinement of purothionins reveals solute particles important for lattice formation and toxicity. Part 2: Structure of beta-purothionin at 1.7 angstroms resolution. Acta Crystallogr, Sect. D 51: 914-924.

Su, W., (Juan, P., and Bugos, R. 2004. High-level secretion of functional green fluorescent protein from transgenic tobacco cell cultures: characterization and sensing. Biotechnology and Bioengineering 85: 610-619.

Terras, F., Schoofs', H., Thevissen, K., Osborn, R., Vanderleyden, J., Cammue, B., and Broekaert, W. F. 1993, Synergistic enhancement of the antifungal activity of wheat and barley thionins by radish and oilseed rape 2s albumins and by barley trypsin inhibitors. Plant Physiol. 103: 1311-1319.

Thevissen, K., Terras, F., and Broekaert, W. F. 1999. Permeability of fungal membranes by plant defensins inhibits fungal growth. Appl. Env. Microbial. 63: 5451-5458.

Thevissen, K., Ghazi, A., De Samblanx, G., Brownlee, C., Osborn, R., Broekaert, W. 1996. Fungal Membrane Responses Induced by Plant Defensins and Thionins. J. Biol. Chem. 271: 15018-15025.

Valueva, T., and Mosolov, V. 2004. Role of inhibitors of proteolytic enzymes in plant defense against phytopathogenic microorganisms, Biochemistry (Mosc) 69: 1305-1309.

Van Campenhout, S., Sagi, L., Vander Stappen, J., and Vlockaert, G. 1998. Characterisation of type-1 thionin loci from the A, B, D, and R genomes of wheat and rye, Theor. Appl. Genetics 96: 80-86.

Vila-Perello, M., Sanchez-Vallee, A., Garcia-Olmedo, F. Molina, A., and Andreu, D, 2005. Structural dissection of a highly knotted peptide reveals minimal motif with antimicrobial activity, J. Biol. Chem. 280: 1661-1668,

Wada, K., Ozaki, Y., Matsubara, H., and Yoshizumi, H. 1982. Studies on purot in by chemical modifications, J. Biochem. 91: 257-263,

Whalen, M., limes, R., Bent, A., and Staskawicz, B. 1991. Identification of Pseudamonas syringae pathogens of Arabidopsis and a bacterial locus determining avirulence on both Arahidopsis and soybean. Plant Cell 3: 49-59.

Zasloff, M. 2002. Antimicrobial peptides of multicellular organisms. Nature 415: 389-395.

What is claimed:

1. A polynucleotide adapted to cause the expression of a thionin in a target plant tissue;
wherein:
(a) said polynucleotide comprises a promoter and a coding sequence, wherein said promoter is operatively linked to said coding sequence;
(b) said promoter is a tissue-appropriate promoter for a target plant tissue or tissues, wherein the target plant tissue or tissues are selected from the group consisting of leaf tissue, root tissue, flower tissue, and fruit tissue;
(c) said coding sequence encodes a peptide comprising a signal peptide domain and a thionin domain;
(d) said thionin domain is identical to a native thionin from a seed from a plant species, or said thionin domain has 85% or greater homology to the amino acid sequence of a native thionin from a seed from a plant species;
(e) said signal peptide is adapted to cause the excretion of said thionin domain from a plant cell, if said polynucleotide should be transcribed and translated in a plant cell; and wherein said signal peptide comprises three motifs: a C-terminal motif, an excretion motif, and an N-terminal motif;
(f) said C-terminal motif consists of from 2 to 7 amino acid residues, comprising one or more acidic amino acid residues, and containing no basic amino acid residues; and wherein, if said polynucleotide should be transcribed and translated in a plant cell, then said C-terminal motif will block the lytic activity of the expressed thionin during transport of the thionin within the cell;
(g) said excretion motif comprises from 10 to 14 nonpolar amino acid residues; said excretion motif is identical to a native excretion motif from a plant signal peptide; and wherein, if said polynucleotide should be transcribed and translated in a plant cell, then said excretion motif is adapted to span the membrane of the plant cell, and thereby to promote the excretion of said thionin through the membrane; and
(h) said N-terminal motif comprises from 4 to 10 amino acid residues, comprising one or more basic amino acid residues; said N-terminal motif is identical to an N-terminal motif from a plant thionin signal peptide that is specific for the same target plant tissue and that is native to the same plant species, or that is specific for the same target plant tissue and that is native to a different plant species; and wherein, if said polynucleotide should be transcribed and translated in a plant cell, then said N-terminal motif is adapted to stabilize the excreted thionin in the plant cell wall, or to inhibit reinsertion of the excreted thionin into the plasmalemma, or both.

2. The polynucleotide of claim 1; wherein said coding sequence encodes a peptide comprising a signal peptide domain, a thionin domain, and a C-terminal acidic peptide domain; and wherein said acidic peptide domain is identical to a native acidic peptide domain associated with a thionin from a plant species, or said acidic peptide domain has 85% or greater homology to a native acidic peptide domain associated with a thionin from a plant species.

3. The polynucleotide of claim 1; wherein said C-terminal motif is identical to a native C-terminal motif from a thionin signal peptide from a plant species.

4. The polynucleotide of claim 1; wherein said thionin domain is identical to a native thionin from a seed from a plant species, but with one or two additional amino acid residues on the N-terminus of said thionin domain as compared to the native thionin.

5. The polynucleotide of claim 1; wherein said polynucleotide is an isolated, recombinant, mutagenized, or synthetic polynucleotide.

6. A transformation vector comprising the polynucleotide of claim 1.

7. A host cell comprising the polynucleotide of claim 1.

8. A method for producing a plant having enhanced resistance to fungal infection, said method comprising transforming plant cells with the polynucleotide of claim 1, wherein the plants cells are capable of regenerating a plant.

9. A plant produced by the method of claim 8, wherein cells of said plant express the encoded thionin.

10. A derivative plant of the plant of claim 9, wherein cells of said derivative plant express the encoded thionin,

11. A seed of the plant of claim 9, or capable of producing the plant of claim 9, wherein cells of said seed comprise said polynucleotide.

12. A seed of the derivative plant of claim 10, or capable of producing the derivative plant of claim 10, wherein cells of said seed comprise said polynucleotide.

13. A method for producing a plant having enhanced resistance to fungal infection, said method comprising crossing or back-crossing the plant of claim 9 with other germplasm to produce a progeny plant, wherein cells of said progeny plant express the encoded thionin

14. A plant produced by the method of claim 13, wherein cells of said plant express the encoded thionin.

15. A derivative plant prepared from the plant of claim 14, wherein cells of said derivative plant express the encoded thionin.

16. A seed of the plant of claim 14, or capable of producing the plant of claim 14, wherein cells of said seed comprise said polynucleotide.

17. A seed of the derivative plant of claim 15, or capable of producing the derivative plant of claim 15, wherein cells of the seed comprise said polynucleotide.

18. The plant of claim 9, wherein said plant is a monocot.

19. The plant of claim 10, wherein said plant is a dicot.


SEQUENCE LISTING (27 sequences)

SEQ ID NO: 1 - DNA, 1297 bp, Artificial sequence
  alpha-HTH gene with SPB signal.  Double CaMV 35S promoter 16 to 555.  Gene 558 to 989.  Hybrid signal peptide SPB 558 to 641. Mature thionin 642 to 776.  Acidic peptide 777 to 965.  His-6 tag 969 to 986. Nos3′ termination region 996 to 1262.
ggtaccaagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgaccatgggaagtatcaaaggtcttaagagtgtagtcgtgtgtttacttatactggggttggttctcgaacaggtgcaagtagaaggcaagagttgctgcaggagcaccctaggaagaaactgctacaatctttgccgcgtccgtggtgctcagaagctatgcgcaggcgtctgtaggtgtaaactcacaagtagcggaaaatgccctacaggcttccccaaattggcccttgtgtccaactcagatgaaccagacaccgtcaagtattgcaacttggggtgtagggcttccatgtgtgactacatggtcaacgcagctgctgacgacgaagaaatgaaactctatttggaaaattgtggtgatgcttgtgtcaatttctgcaacggtgatgctggcctcacatccctttctagacaccatcaccatcaccattaacccgggcagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggaattcactagtgagtcgtattacgtagct

SEQ ID NO: 2 - protein, 143 aa, Artificial sequence
  alpha-HTH peptide with SPB signal peptide. Artificial alpha-HTH precursor, positions 1 to 143.  Hybrid signal peptide SPB, positions 1 to 28.  Mature thionin, positions 29 to 73.  Acidic peptide, positions 74 to 136.  His-6 tag, positions 139 to 143.
MGSIKGLKSVVVCLLILGLVLEQVQVEGKSCCRSTLGRNCYNLCRVRGAQKLCAGVCRCKLTSSGKCPTGFPKLALVSNSDEPDTVKYCNLGCRASMCDYMVNAAADDEEMKLYLENCGDACVNFCNGDAGLTSLSRHHHHHH

SEQ ID NO: 3 - DNA, 84 bp, Artificial sequence
  Hybrid signal SPB = preferred signal sequence. Hybrid signal peptide SPB, positions 1 to 84.  N-terminus of oat Asthi1 signal peptide, positions 1 to 33.  Alpha-HTH signal peptide (no methionine), positions 34 to 84.
atgggaagtatcaaaggtcttaagagtgtagtcgtgtgtttacttatactggggttggttctcgaacaggtgcaagtagaaggc

SEQ ID NO: 4 - protein, 28 aa, Artificial sequence
  Hybrid signal peptide SPB = preferred signal peptide sequence. Hybrid signal peptide SPB, positions 1 to 28. N-terminus of oat Asthi1 signal peptide, positions 1 to 11. Alpha-HTH signal peptide (no methionine), positions 12 to 28.
MGSIKGLKSVVVCLLILGLVLEQVQVEG

SEQ ID NO: 5 - protein, 28 aa, Hordeum vulgare
  misc_feature: Barley leaf-specific thionin DB4.
MAPSKSIKSVVICVLILGLVLEQVQVEG

SEQ ID NO: 6 - protein, 28 aa, Hordeum vulgare
  misc_feature: Barley leaf-specific thionin BTH6.
MAPSKSIKSVVICVLILVLVLEQVQVEG

SEQ ID NO: 7 - protein, 28 aa, Avena sativa
  misc_feature: Avena sativa
MGSIKGLKSVVICVLVLGIVLEQVQVEG

SEQ ID NO: 8 - protein, 24 aa, Arabidopsis thaliana
  misc_feature: Arabidopsis leaf-specific thionin Thi2.1.
MKGRILILSLLIMSLVMAQVQVEA

SEQ ID NO: 9 - protein, 24 aa, Arabidopsis thaliana
  misc_feature: Arabidopsis leaf-specific thionin Thi2.2.
MEGKTVISSLLIMSLVLAQIQVEA

SEQ ID NO: 10 - DNA, 18 bp, Artificial sequence
  PrGRthi primer
cctcctagatctcaagag

SEQ ID NO: 11 - DNA, 39 bp, Artificial sequence
  PrthioB1 primer
ctttccatgcgaagtatcaaaggtcttaagagtgtagtc

SEQ ID NO: 12 - DNA, 29 bp, Artificial sequence
  PrthioC1 primer
ctttccatggggatccaaggagatataac

SEQ ID NO: 13 - DNA, 27 bp, Artificial sequence
  LnT0504 primer
ggatccaccatcaccatcaccattgca

SEQ ID NO: 14 - DNA, 49 bp, Artificial sequence
  PrrthioA2 primer
ctttcccgggttaatgatgatgatgatgatgtctagaaagggatgtgag

SEQ ID NO: 15 - DNA, 27 bp, Artificial sequence
  LnrT0504 primer
atggtgatggtgatggtggatcctgca

SEQ ID NO: 16 - DNA, 1300 bp, Artificial sequence
  alpha-HTH gene with SPA signal.  Double CaMV 35S promoter 16-555. Gene 558-986.  rice glycine rich protein signal peptide SPA 558-638.  Linker  639-644.  Mature thionin 639-779.  Acidic peptide 780-968.  His-6 tag 972-989.  Nos3′ termination region
ggtaccaagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgaccatggctactactaagcatttggctcttgccatccttgtcctccttagcattggtatgaccaccagtgcaagaaccctcctagatctcaagagttgctgcaggagcaccctaggaagaaactgctacaatctttgccgcgtccgtggtgctcagaagctatgcgcaggcgtctgtaggtgtaaactcacaagtagcggaaaatgccctacaggcttccccaaattggcccttgtgtccaactcagatgaaccagacaccgtcaagtattgcaacttggggtgtagggcttccatgtgtgactacatggtcaacgcagctgctgacgacgaagaaatgaaactctatttggaaaattgtggtgatgcttgtgtcaatttctgcaacggtgatgctggcctcacatccctttctagacaccatcaccatcaccattaacccgggcagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggaattcactagtgagtcgtattacgtagct

SEQ ID NO: 17 - protein, 144 aa, Artificial sequence
  alpha-HTH peptide with SPA signal peptide. Artificial alpha-HTH precursor 1-144.  rice glycine rich protein signal peptide SPA 1-27.  Linker 28-29.  Mature thionin 30-74. Acidic peptide, positions 75-136.  His-6 tag 139-144.
MATTKHLALAILVLLSIGMTTSARTLLDLKSCCRSTLGRNCYNLCRVRGAQKLCAGVCRCKLTSSGKCPTGFPKLALVSNSDEPDTVKYCNLGCRASMCDYMVNAAADDEEMKLYLENCGDACVNFCNGDAGLTSLSRHHHHHH

SEQ ID NO: 18 - DNA, 1300 bp, Artificial sequence
  alpha-HTH gene with SPC signal.  Double CaMV 35S promoter 16-555. Gene 582-986.  Arabidopsis basic chitinase signal peptide SPC 582-644.  Mature thionin 645-779.  Acidic peptide 780-968.  His-6 tag 972-989.  Nos3′ termination region 999-1265.
ggtaccaagcttcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcttgaccatggggatccaaggagatataacaatgaagactaatctttttctctttctcatcttttcacttctcctatcattatcctcggccgaaaagagttgctgcaggagcaccctaggaagaaactgctacaatctttgccgcgtccgtggtgctcagaagctatgcgcaggcgtctgtaggtgtaaactcacaagtagcggaaaatgccctacaggcttccccaaattggcccttgtgtccaactcagatgaaccagacaccgtcaagtattgcaacttggggtgtagggcttccatgtgtgactacatggtcaacgcagctgctgacgacgaagaaatgaaactctatttggaaaattgtggtgatgcttgtgtcaatttctgcaacggtgatgctggcctcacatccctttctagacaccatcaccatcaccattaacccgggcagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggaattcactagtgagtcgtattacgtagct

SEQ ID NO: 19 - protein, 136 aa, Artificial sequence
  alpha-HTH peptide with SPC signal peptide. Artificial alpha-HTH precursor 1-136.  Arabidopsis basic chitinase signal peptide SPC 1-21.  Mature thionin 22-66.  Acidic peptide 67-129.  His-6 tag 131-136.
MKTNLFLFLIFSLLLSLSSAEKSCCRSTLGRNCYNLCRVRGAQKLCAGVCRCKLTSSGKCPTGFPKLALVSNSDEPDTVKYCNLGCRASMCDYMVNAAADDEEMKLYLENCGDACVNFCNGDAGLTSLSRHHHHHH

SEQ ID NO: 20 - protein, 109 aa, Triticum aestivum
  misc_feature: Portion of beta-purothionin from wheat seed. Portion of beta-purothionin precursor, positions 1 to 109. Mature thionin, positions 1 to 45.  Acidic peptide, positions 46 to 109.
KSCCKSTLGRNCYNLCRARGAQKLCANVCRCKLTSGLSCPKDFPKLVLESNSDEPDTMEYCNLGCRSSLCDYIVNAAADDEEMKLYVEQCGDACVNFCNADAGLTSLDA

SEQ ID NO: 21 - protein, 109 aa, Triticum aestivum
  misc_feature: Portion of alpha-1-purothionin from wheat seed. Portion of alpha-1-purothionin precursor, positions 1 to 109. Mature thionin, positions 1 to 45.  Acidic peptide, positions 46 to 109.
KSCCRTTLGRNCYNLCRSRGAQKLCSTVCRCKLTSGLSCPKGFPKLALESNSDEPDTIEYCNLGCRSSVCDYMVNAAADDEEMKLYVENCGDACVNFCNGDAGLTSLDA

SEQ ID NO: 22 - protein, 109 aa, Hordeum vulgare
  misc_feature: Portion of beta-hordothionin from wheat seed. Portion of beta-hordothionin precursor, positions 1 to 109. Mature thionin, positions 1 to 45.  Acidic peptide, positions 46 to 109.
KSCCRSTLGRNCYNLCRVRGAQKLCANACRCKLTSGLKCPSSFPKLALVSNSDEPDTIDYCNLGCRASMCDYMVNAAADDEEMKLYVEHCSDACVNFCNGDVGLTSLTA

SEQ ID NO: 23 - protein, 68 aa, Hordeum vulgare
  misc_feature: alpha-HTH.
GLKGVMVCLLILGLVLEQVQVEGKSCCRSTLGRNCYNLCRVRGAQKLCAGVCRCKLTSSGKCPTGFPK

SEQ ID NO: 24 - protein, 72 aa, Triticum aestivum
  misc_feature: beta-PTH.
MGSKGLKGVMVCLLILGLVLEQVQVEGKSCCKSTLGRNCYNLCRARGAQKLCANVCRCKLTSGLSCPKDFPK

SEQ ID NO: 25 - protein, 74 aa, Hordeum vulgare
  misc_feature: BTH6.
MAPSKSIKSVVICVLILVLVLEQVQVEGKSCCKDTLARNCYNTCRFAGGSRPVCAGACRCKIISGPKCPSDYPK

SEQ ID NO: 26 - protein, 74 aa, Avena sativa
  misc_feature: Asthil.
MGSIKGLKSVVICVLVLGIVLEQVQVEGKSCCKDIMARNCYNVCRIPGTPRPVCATTCRCKIISGNKCPKDYPK

SEQ ID NO: 27 - protein, 67 aa, Arabidopsis thaliana
  misc_feature: Thi2.1.
MKGRILILSLLIMSLVMAQVQVEAKICCPSNQARNGYSVCRIRFSKGRCMQVSGCQNSDTCPRGWVN